(19) United States
(12) Patent Application Publication
(10) Pub. No.: US 2007/0178140 A1
(43) Pub. Date: Aug. 2, 2007

(54) COMPOSITIONS AND METHODS FOR REDUCING FOOD INTAKE AND CONTROLLING WEIGHT

(76) Inventors: William Ronald Aimutis JR., Blaine, MN (US); Janet Deihl, Doylestown, PA (US); Teresa Marie Paeschke, Minneapolis, MN (US); Timothy E. Tracy, Pennington, NJ (US); Thomas E. Sox, Ambler, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/539,244

(22) Filed: Oct. 6, 2006

Related U.S. Application Data
(63) Continuation-in-part of application No. 11/245,763, filed on Oct. 7, 2005.

(51) Int. Cl. A61K 47/00

(52) U.S. Cl. 424/439

(57) ABSTRACT

A formed food or other ingestible composition having at least one soluble anionic fiber and at least one multivalent cation wherein the combination of fiber and cation increases viscosity of digesta. Methods of reducing blood glucose levels or insulin levels are disclosed. Also disclosed are methods of controlling, ameliorating, or treating diabetes with ingestible compositions comprising a combination of soluble anionic fiber and at least one multivalent cation.

### CROSS-REFERENCE TO RELATED APPLICATIONS

This case is related to U.S. patent application Ser. No. 11/245,763, entitled ‘COMPOSITIONS AND METHODS FOR REDUCING FOOD INTAKE AND CONTROLLING WEIGHT” (docket number MSP5038); U.S. patent application Ser. No. 11/245,874, entitled ‘COMPOSITIONS AND METHODS FOR REDUCING FOOD INTAKE AND CONTROLLING WEIGHT” (docket number MSP5039); U.S. patent application Ser. No. 11/245,910, entitled “COMPOSITIONS AND METHODS FOR INDUCING SATIETY AND REDUCING CALORIC INTAKE” (docket number MSP5040); U.S. patent application Ser. No. 11/245,762, entitled “METHODS FOR ACHIEVING AND MAINTAINING WEIGHT LOSS” (docket number MSP5041); U.S. patent application Ser. No. 11/245,832, entitled “METHODS FOR REDUCING CALORIE INTAKE” (docket number MSP5042), U.S. patent application Ser. No. 11/245,872, entitled “COMPOSITIONS AND METHODS FOR REDUCING FOOD INTAKE AND CONTROLLING WEIGHT” (docket number MSP5043); U.S. patent application Ser. No. 11/245,798, entitled “COMPOSITIONS AND METHODS FOR REDUCING FOOD INTAKE AND CONTROLLING WEIGHT” (docket number MSP5044); U.S. patent application Ser. No. 11/245,621, entitled “METHODS FOR WEIGHT MANAGEMENT” (docket number MSP5045); U.S. patent application Ser. No. 11/245,869, entitled “METHODS FOR INDUCING SATIETY, REDUCING FOOD INTAKE AND REDUCING WEIGHT” (docket number MSP5046); U.S. patent application Ser. No. 11/245,873, entitled “COMPOSITIONS AND METHODS FOR REDUCING FOOD INTAKE AND CONTROLLING WEIGHT (docket number MSP5047); U.S. patent application Ser. No. 11/246,938, entitled “FIBER SATIETY COMPOSITIONS” (docket number SPC5 185); and U.S. patent application Ser. No. 11/246,646, entitled “FIBER SATIETY COMPOSITIONS” (docket number SPC5186), each filed concurrently on Oct. 7, 2005, and U.S. patent application Ser. No. ______, entitled “CONTROLLED HYDRATION OF HYDROCOLLOIDS” (docket number 10790-065001) filed concurrently herewith on Oct. 6, 2006.

### FIELD OF THE INVENTION

The present invention is directed to formed foods that include at least one soluble anionic fiber and at least one multivalent cation and methods for inducing satiety in an animal, reducing caloric intake in an animal, reducing weight in an animal, improving weight reduction in an animal, and decreasing blood glucose and insulin levels using the ingestible compositions.

### BACKGROUND OF THE INVENTION

Diabetes and obesity are common ailments in the United States and other Western cultures. A study by researchers at RTI International and the Centers for Disease Control estimated that U.S. obesity-attributable medical expenditures reached $75 billion in 2003. Obesity has been shown to promote many chronic diseases, including type 2 diabetes, cardiovascular disease, several types of cancer, and gallbladder disease.

Adequate dietary intake of soluble fiber has been associated with a number of health benefits, including decreased blood cholesterol levels, improved glycemic control, and the induction of satiety and satiation in individuals. Consumers have been resistant to increasing soluble fiber amounts in their diet due to the negative organoleptic characteristics, such as, sliminess, excessive viscosity, excessive dryness and poor flavor, that are associated with food products that include soluble fiber.

What is needed is a stable, organoleptically acceptable food product that delivers a cation and at least one soluble anionic fiber where the cation will react with the anionic fiber to create a viscous material in vivo, but do not react in vitro over the shelf life of the product.

### SUMMARY OF THE INVENTION

The present invention solves the above needs by providing a formed food comprising, consisting of, and/or consisting essentially of an effective amount of at least one soluble anionic fiber and an effective amount of at least one multivalent cation wherein the combination of fiber and cation increases viscosity of digesta.

Another embodiment of the present invention is an ingestible composition comprising consisting of, and/or consisting essentially of a solid phase comprising, consisting of, and/or consisting essentially of at least one soluble anionic fiber in a total amount of from about 0.5 g to about 10 g per serving and a fluid phase in intimate contact with the solid phase, the fluid phase comprising, consisting of, and/or consisting essentially of calcium in an amount of from about 50 to about 300 mg of elemental calcium per serving.

A further embodiment of the present invention is directed to a method for inducing satiety in an animal, the method comprising, consisting of, and/or consisting essentially of the step of orally administering to the animal a serving of a formed food comprising at least one soluble anionic fiber and at least one multivalent cation.

Another embodiment of the present invention is a method for inducing satiety in an animal, the method comprising, consisting of, and/or consisting essentially of the step of orally administering to the animal a serving of a food comprising, consisting of, and/or consisting essentially of a formed solid phase comprising at least one soluble anionic fiber in a total amount of from about 0.5 g to about 10 g per serving and a fluid phase in intimate contact with the formed solid phase, the fluid phase comprising calcium in an amount of from about 50 to about 500 mg of elemental calcium per serving.

A still further embodiment of the present invention is a method for reducing caloric intake in an animal, the method comprising, consisting of, and/or consisting essentially of the step of orally administering to the animal a serving of a formed food comprising, consisting of, and/or consisting essentially of at least one soluble anionic fiber and at least one multivalent cation.

An additional embodiment of the present invention is a method for reducing caloric intake in an animal, the method comprising, consisting of, and/or consisting essentially of the step of orally administering to the animal a serving of an food comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the formed solid phase, the fluid phase comprising, consisting of, and/or consisting essentially of calcium in an amount of from about 50 to about 300 mg of elemental calcium per serving.

A further additional embodiment of the present invention is a method for reducing weight in an animal, the method comprising, consisting of, and/or consisting essentially of the step of orally administering to the animal a serving of a formed food comprising, consisting of, and/or consisting essentially of at least one soluble anionic fiber and at least one multivalent cation.

Another embodiment of the present invention is a method for reducing weight in an animal, the method comprising, consisting of, and/or consisting essentially of the step of orally administering to the animal a serving of an ingestible composition comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the formed solid phase, the fluid phase comprising, consisting of, and/or consisting essentially of calcium in an amount of from about 50 to about 300 mg of elemental calcium per serving.

A further embodiment of the present invention is a method for improving weight reduction by at least 5% in an animal, the method comprising, consisting of, and/or consisting essentially of the step of orally administering to the animal a serving of a formed food comprising, consisting of, and/or consisting essentially of at least one soluble anionic fiber and a multivalent cation, wherein the weight reduction improvement is measured after four months of daily administration of the ingestible composition.

A still further embodiment of the present invention is a method for improving weight reduction by at least 5% in an animal, the method comprising, consisting of, and/or consisting essentially of the step of orally administering to the animal an ingestible composition comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the formed solid phase, the fluid phase comprising calcium in an amount of from about 50 to about 300 mg of elemental calcium per serving, wherein the weight reduction improvement is measured after four months of daily administration of the ingestible composition.

An additional embodiment of the present invention is a method for decreasing postprandial glucose and insulin levels after a meal, the method comprising, consisting of, and/or consisting essentially of orally administering to the animal an ingestible composition comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the formed solid phase, the fluid phase comprising calcium in an amount of from about 50 to about 300 mg of elemental calcium per serving, wherein the ingestible composition is consumed from about two hours before the meal to about two hours after the meal.

A further embodiment of the present invention is a method for slowing the development or progression of diabetes, the method comprising, consisting of, and/or consisting essentially of orally administering to the animal an ingestible composition comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the formed solid phase, the fluid phase comprising calcium in an amount of from about 50 to about 300 mg of elemental calcium per serving, wherein the ingestible composition is consumed from about two hours before the meal to about two hours after the meal.

An additional embodiment of the present invention is an ingestible composition comprising, consisting of, and/or consisting essentially of a formed solid phase comprising, consisting of, and/or consisting essentially of at least one soluble anionic fiber in a total amount of from about 0.5 g to about 10 g per serving and a fluid phase in intimate contact with the baked phase, the fluid phase comprising, consisting of, and/or consisting essentially of calcium in an amount of from about 50 to about 300 mg of elemental calcium per serving.

### BRIEF DESCRIPTION OF DRAWINGS

FIG. 1 is a graph depicting the effects of an embodiment of the present invention on intestinal viscosity.

FIG. 2 is a graph depicting the effects of an embodiment of the present invention on postprandial glucose levels.

FIG. 3 is a graph depicting the effects of an embodiment of the present invention on postprandial insulin levels.

FIG. 4 is graph depicting the effects of another embodiment of the present invention on postprandial glucose levels.

FIG. 5 is a graph depicting the effects of another embodiment of the present invention on postprandial insulin levels.

### DETAILED DESCRIPTION OF THE INVENTION

As used herein, unless indicated otherwise, the terms “alginate,” “pectin,” “carrageenan,” “polygeenan,” or “gellan” refers to all forms (e.g., protonated or salt forms, such as sodium, potassium, and ammonium salt forms and having varying average molecular weight ranges) of the soluble anionic fiber type.

As used herein, unless indicated otherwise, the term “alginic acid” includes not only the material in protonated form but also the related salts of alginate, including, but not limited, to sodium, potassium, and ammonium alginate.

As used herein, unless indicated otherwise, the term “protected” means that the source has been treated in such a way, as illustrated below, to delay (e.g., until during or after ingestion or until a certain pH range has been reached) reaction of the at least one multivalent cation with the soluble anionic fiber as compared to an unprotected multivalent cation.

As used herein, unless indicated otherwise, the term “insulin response” means the increase in blood insulin levels that results from the oral intake of nutritional materials, particularly glucose and glucose-containing carbohydrates.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, a recitation of a range of values is merely intended to serve as a shorthand method of referring individually to each separate value falling within the range, and each separate value is incorporated into the specification as if it were individually recited herein.

Soluble Anionic Fiber

Any soluble anionic fiber should be acceptable for the purposes of this invention. Suitable soluble anionic fibers include alginate, pectin, gellan, soluble fibers that contain carboxylate substituents, carrageenan, polygeenan, and marine algae-derived polymers that contain sulfate substituents.

Also included within the scope of soluble anionic fibers are other plant derived and synthetic or semisynthetic polymers that contain sufficient carboxylate, sulfate, or other anionic moieties to undergo gelling in the presence of sufficient levels of multivalent cation.

At least one source of soluble anionic fiber may be used in these compositions, and the at least one source of soluble anionic fiber may be combined with at least one source of soluble fiber that is uncharged at neutral pH. Thus, in certain cases, two or more soluble anionic fibers types are included, such as, alginate and pectin, alginate and gellan, or pectin and gellan. In other cases, only one type of soluble anionic fiber is used, such as only alginate, only pectin, only carrageenan, or only gellan.

Soluble anionic fibers are commercially available, e.g., from ISP (Wayne, N.J.), TIC Gums, and CP Kelco.

An alginate can be a high guluronic acid alginate. For example, in certain cases, an alginate can exhibit a higher than 1:1 ratio of guluronic to mannuronic acids, such as in the range from about 1.2:1 to about 1.8:1, e.g., about 1.3:1, about 1.4:1, about 1.5:1, about 1.6:1, or about 1.7:1 or any value therebetween. Examples of high guluronic alginates (e.g., having a higher than 1:1 g:m ratios) include Manugel LBA, Manugel GHB, and Manugel DBP, which each have a g:m ratio of about 1.5.

While not being bound by theory, it is believed that high guluronic alginates can cross-link through multivalent cations, e.g., calcium ions, to form gels at the low pH regimes in the stomach. High guluronic alginates are also believed to electrostatically associate with pectins and/or gellans at low pHs, leading to gelation. In such cases, it may be useful to delay the introduction of multivalent cations until after formation of the mixed alginate/pectin or alginate/gellan gel, as multivalent cationic cross-links may stabilize the mixed gel after formation.

In other cases, an alginate can exhibit a ratio of guluronic to mannuronic acids (g:m ratio) of less than about 1:1, e.g., about 0.8:1 to about 0.4:1, such as about 0.5:1, about 0.6:1, or about 0.7:1 or any value therebetween. Keltone LV and Keltone HV are examples of high-mannuronic acids (e.g., having a g:m ratio of less than 1:1) having g:m ratios ranging from about 0.6:1 to about 0.7:1.

Methods for measuring the ratio of guluronic acids to mannuronic acids are known by those having ordinary skill in the art.

An alginate can exhibit any number average molecular weight range, such as a high molecular weight range (about 2.05×10⁵ to about 3×10⁵ Daltons or any value therebetween; examples include Manugel DPB, Keltone HV, and TIC 900 Alginate); a medium molecular weight range (about 1.38×10⁵ to about 2×10⁵ Daltons or any value therebetween; examples include Manugel GHB); or a low molecular weight range (2×10⁴ to about 1.35×10⁵ Daltons or any value therebetween; examples include Manugel LBA and Manugel LBB). Number average molecular weights can be determined by those having ordinary skill in the art, e.g., using size exclusion chromatography (SEC) combined with refractive index (RI) and multi-angle laser light scattering (MALLS).

In certain embodiments of a formed food product, a low molecular weight alginate can be used (e.g., Manugel LBA), while in other cases a mixture of low molecular weight (e.g., Manugel LBA) and high molecular weight (e.g., Manugel DPB, Keltone HV) alginates can be used. In other cases, a mixture of low molecular weight (e.g., Manugel LBA) and medium molecular weight (e.g., Manugel GHB) alginates can be used. In yet other cases, one or more high molecular weight alginates can be used (e.g., Keltone HV, Manugel DPB).

A pectin can be a high-methoxy pectin (e.g., having greater than 50% esterified carboxylates), such as ISP HM70LV and CP Kelco USPL200. A pectin can exhibit any number average molecular weight range, including a low molecular weight range (about 1×10⁵ to about 1.20×10⁵ Daltons, e.g., CP Kelco USPL200), medium molecular weight range (about 1.25×10⁵ to about 1.45×10^{5,} e.g., ISP HM70LV), or high molecular weight range (about 1.50×10⁵ to about 1.80×10^{5,} e.g., TIC HM Pectin). In certain cases, a high-methoxy pectin can be obtained from pulp, e.g., as a by-product of orange juice processing.

A gellan soluble anionic fiber can also be used. Gellan fibers form strong gels at lower concentrations than alginates and/or pectins, and can cross-link with multivalent cation cations. For example, gellan can form gels with magnesium and calcium. Gellans for use in the invention include Kelcogel, available commercially from CP Kelco.

A range of fiber intake in the compositions of this invention may be about 0.25 g to about 5 g per serving, more preferably about 0.5 to about 3 g per serving, and most preferably about 1.0 to about 2.0 g per serving.

Fiber blends as described herein can also be used in the preparation of a solid ingestible composition like an extruded food product where the fiber blend is a source of the soluble anionic fiber. A useful fiber blend can include an alginate soluble anionic fiber and a pectin soluble anionic fiber. A ratio of total alginate to total pectin in a blend can be from about 8:1 to about 5:1, or any value therebetween, such as about 7: 1, about 6.5: 1, about 6.2: 1, or about 6.15:1. A ratio of a medium molecular weight alginate to a low molecular weight alginate can range from about 0.65:1 to about 2:1, or any value therebetween.

An alginate soluble anionic fiber in a blend may be a mixture of two or more alginate forms, e.g., a medium and low molecular weight alginate. In certain cases, a ratio of a medium molecular weight alginate to a low molecular weight alginate is about 0.8:1 to about 0.9:1. The fiber blend combining low and medium molecular weight alginates with high methoxy pectin has been tested at about 0 to about 3 grams. A preferred range for both may be about 1 to about 2 grams.

The at least one soluble anionic fiber may be treated before, during, or after incorporation into an ingestible composition. For example, the at least one soluble anionic fiber can be processed, e.g., extruded, roll-dried, freeze-dried, dry blended, roll-blended, agglomerated, coated, or spray-dried.

For solid forms, a variety of formed food products can be prepared by methods known to those having ordinary skill in the art, e.g., extruding, molding, pressing, wire-cutting, and the like. For example, a single or double screw extruder can be used. Typically, a feeder meters in the raw ingredients to a barrel that includes the screw(s). The screw(s) conveys the raw material through the die that shapes the final product. Extrusion can take place under high temperatures and pressures or can be a non-cooking, forming process. Extruders are commercially available, e.g., from Buhler, Germany. Extrusion can be cold or hot extrusion.

Other processing methods are known to those having skilled in the art.

The amount of the at least one soluble anionic fiber included can vary, and will depend on the type of ingestible composition and the type of soluble anionic fiber used. For example, typically an ingestible composition, e.g., formed food, will include from about 0.5 g to about 10 g total soluble anionic fiber per serving or any value therebetween. In certain cases, a formed food product may include an soluble anionic fiber at a total amount from about 22% to about 40% by weight of the formed food product or any value therebetween. In other cases, a formed food product may include an soluble anionic fiber in a total amount of from about 4% to about 15% or any value therebetween, such as when only gellan is used. In yet other cases, a formed food product can include an soluble anionic fiber at a total amount of from about 18% to about 25% by weight, for example, when combinations of gellan and alginate or gellan and pectin are used.

In addition to the at least one soluble anionic fiber, a solid ingestible composition can include ingredients that may be treated in a similar manner as the at least one soluble anionic fiber. For example, such ingredient can be co-extruded with the soluble anionic fiber, co-processed with the soluble anionic fiber, or co-spray-dried with the soluble anionic fiber. Such treatment can help to reduce sliminess of the ingestible composition in the mouth and to aid in hydration and gelation of the fibers in the stomach and/or small intestine. Without being bound by any theory, it is believed that co-treatment of the soluble anionic fiber(s) with such ingredient prevents early gelation and hydration of the fibers in the mouth, leading to sliminess and unpalatability. In addition, co-treatment may delay hydration and subsequent gelation of the soluble anionic fibers (either with other soluble anionic fibers or with multivalent cations) until the ingestible composition reaches the stomach and/or small intestine, providing for the induction of satiety and/or satiation.

Additional ingredients can be hydrophilic in nature, such as, starch, protein, maltodextrin, and inulin. Other additional ingredients can be insoluble in water (e.g., cocoa solids, corn fiber) and/or fat soluble (vegetable oil), or can be flavor modifiers, such as, sucralose. For example, a formed food product can include from about 5 to about 80% of a cereal ingredient, such as, about 40% to about 68% of a cereal ingredient. A cereal ingredient can be rice, corn, wheat, sorghum, oat, or barley grains, flours, or meals. Thus, an extruded food product can include about 40% to about 50%, about 50% to about 58%, about 52% to about 57%, or about 52%, about 53%, about 54%, about 55%, about 56%, or about 56.5% of a cereal ingredient. In one embodiment, about 56.5% of rice flour is included.

A formed food may also include a protein source. A protein source may be included in the composition. For example, an extruded food product can include a protein source at about 2% to about 20% by weight, such as about 3% to about 8%, about 3% to about 5%, about 4% to about 7%, about 4% to about 6%, about 5% to about 7%, about 5% to about 15%, about 10% to about 18%, about 15% to about 20%, or about 8% to about 18% by weight. A protein can be any known to those having ordinary skill in the art, e.g., rice, milk, egg, wheat, whey, soy, gluten, or soy flour. In some cases, a protein source can be a concentrate or isolate form.

Multivalent Cation

The compositions and associated methods of this invention include a source of at least one multivalent, e.g., divalent cation in an amount sufficient to cause an increase in viscosity of the digesta of an animal. A source of at least one multivalent cation may be incorporated into an ingestible composition provided herein, or can consumed as a separate food article either before, after, or simultaneously with an ingestible composition.

Any multivalent cation may be used in the present invention, e.g., divalent, trivalent, and the like. Multivalent cations useful in this invention include, calcium, magnesium, aluminum, manganese, their salts and mixtures thereof. Salts of the multivalent cations may be organic acid salts that include formate, fumarate, acetate, propionate, butyrate, caprylate, valerate, lactate, citrate, malate and gluconate. Also included are highly soluble inorganic salts such as chlorides or other halide salts.

In certain compositions, one or more particular multivalent cations may be used with certain soluble anionic fibers, depending on the composition and gel strength desired. For example, for ingestible alginate compositions, calcium may be used to promote gelation. For gellan compositions, one or more of calcium and magnesium may be used.

The at least one multivalent cation can be unable to, or be limited in its ability to, react with the at least one soluble anionic fiber in the ingestible composition until during or after ingestion. For example, physical separation of the at least one multivalent cation from the at least one soluble anionic fiber, e.g., as a separate food article or in a separate matrix of the ingestible composition from the at least one soluble anionic fiber, can be used to limit at least one multivalent cation's ability to react. In other cases, the at least one multivalent cation is limited in its ability to react with the at least one soluble anionic fiber by protecting the source of at least one multivalent cation until during or after ingestion. Thus, the at least one multivalent cation, such as, a protected multivalent cation, can be included in the ingestible composition or can be included as a separate food article composition, e.g., for separate ingestion either before, during, or after ingestion of an ingestible composition.

Typically, a separate food article containing the source of at least one multivalent cation would be consumed in an about four hour time window flanking the ingestion of an ingestible composition containing the at least one soluble anionic fiber. In certain cases, the window may be about three hours, or about two hours, or about one hour. In other cases, the separate food article may be consumed immediately before or immediately after ingestion of an ingestible composition, e.g., within about fifteen minutes, such as within about 10 minutes., about 5 minutes, or about 2 minutes. In other cases, a separate food article containing at least one multivalent cation can be ingested simultaneously with an ingestible composition containing the at least one soluble anionic fiber, e.g., a snack chip composition where some chips include at least one multivalent cation and some chips include the at least one soluble anionic fiber.

In one embodiment, at least one multivalent cation can be included in an ingestible composition in a different food matrix from a matrix containing an soluble anionic fiber. For example, a source of at least one multivalent cation, such as a calcium salt, can be included in a separate matrix of a solid ingestible composition from the matrix containing the at least one soluble anionic fibers. Thus, means for physical separation of an soluble anionic fiber (e.g., within a snack bar or other extruded food product) from a source of at least one multivalent cation are also contemplated, such as by including the source of at least one multivalent cation in a matrix such as a frosting, water and fat based icing, coating, decorative topping, drizzle, chip, chunk, swirl, filling, or interior layer. In one embodiment, a source of at least one multivalent cation, such as a protected multivalent cation source, can be included in a snack bar matrix that also contains an extruded crispy matrix that contains the soluble anionic fiber. In such a case, the source of at least one multivalent cation is in a separate matrix than the extruded crispy matrix containing the soluble anionic fiber. In another embodiment, a source of at least one multivalent cation can be included in a gel layer or phase, e.g., a jelly or jam.

One multivalent cation source is multivalent cation salts. Typically, a multivalent cation salt can be selected from the following salts: citrate, tartrate, malate, formate, lactate, gluconate, phosphate, carbonate, sulfate, chloride, acetate, propionate, butyrate, caprylate, valerate, fumarate, adipate, and succinate. In certain cases, a multivalent cation salt is a calcium salt. A calcium salt can have a solubility of >1% w/vol in water at pH 7 at 20° C. A calcium salt can be, without limitation, calcium citrate, calcium tartrate, calcium malate, calcium lactate, calcium gluconate, dicalcium phosphate dihydrate, anhydrous calcium diphosphate, calcium citrate malate, dicalcium phosphate anhydrous, calcium carbonate, calcium sulfate dihydrate, calcium sulfate anhydrous, calcium chloride, calcium acetate monohydrate, monocalcium phosphate monohydrate, and monocalcium phosphate anhydrous.

The source of at least one multivalent cation can be a protected source.

A number of methods can be used to protect a source of at least one multivalent cation. For example, microparticles or nanoparticles having double or multiple emulsions, such as water/oil/water (“w/o/w”) or oil/water/oil (“o/w/o”) emulsions, of at least one multivalent cation and an soluble anionic fiber can be used. In one embodiment, a calcium alginate microparticle or nanoparticle is used. For example, a calcium chloride solution can be emulsified in oil, which emulsion can then be dispersed in a continuous water phase containing the anionic alginate soluble fiber. When the emulsion breaks in the stomach, the calcium can react with the alginate to form a gel.

A microparticle can have a size from about 1 to about 15 μM (e.g., about 5 to about 10 μM, or about 3 to about 8 μM). A nanoparticle can have a size of about 11 to about 85 nm (e.g., about 15 to about 50 nm, about 30 to about 80 nm, or about 50 to about 75 nm). The preparation of multiple or double emulsions, including the choice of surfactants and lipids, is known to those having ordinary skill in the art.

In another embodiment, nanoparticles of calcium alginate are formed by preparing nanodroplet w/o microemulsions of CaCl₂ in a solvent and nanodroplet w/o microemulsions of alginate in the same solvent. When the two microemulsions are mixed, nanoparticles of calcium alginate are formed. The particles can be collected and dispersed, e.g., in a fluid ingestible composition. As the particle size is small (<100 nm), the particles stay dispersed (e.g., by Brownian motion), or can be stabilized with a food grade surfactant. Upon ingestion, the particles aggregate and gel.

In other embodiments, a liposome containing a source of at least one multivalent cation can be included in an ingestible composition. For example, a calcium-containing liposome can be used. The preparation of liposomes containing multivalent cations is well known to those having ordinary skill in the art; see ACS Symposium Series, 1998 709:203-211; Chem. Mater. 1998 (109-116). Cochelates can also be used, e.g., as described in U.S. Pat. No. 6,592,894 and U.S. Pat. No. 6,153,217. The creation of cochelates using multivalent cations such as calcium can protect the multivalent cations from reacting with the soluble anionic fiber within the aqueous phase of an ingestible composition, e.g., by wrapping the multivalent cations in a hydrophobic lipid layer, thus delaying reaction with the fiber until digestion of the protective lipids in the stomach and/or small intestine via the action of lipases.

In certain cases, a multivalent cation-containing carbohydrate glass can be used, such as a calcium containing carbohydrate glass. A carbohydrate glass can be formed from any carbohydrate such as, without limitation, sucrose, trehalose, inulin, maltodextrin, corn syrup, fructose, dextrose, and other mono-, di-, or oligosaccharides using methods known to those having ordinary skill in the art; see, e.g., WO 02/05667. A carbohydrate glass can be used, e.g., in a coating or within a food matrix.

Proteins can also be used to encapsultate the multivalent cation.

Ingestible Compositions

Compositions of the present invention include formed foods and ingestible compositions having a solid phase and a fluid phase.

Compositions of the present invention include bread, cracker, bar, mini-bars, cookie, confectioneries, e.g., nougats, toffees, fudge, caramels, hard candy enrobed soft core, icings, frostings, fruit leathers, muffins, cookies, brownies, cereals, chips, snack foods, bagels, chews, crispies, and nougats, pudding, jelly, and jam. Compositions of the present invention can have densities from low to high.

Fluids

A fluid phase can be useful for, among other things, delivering about 1 g, 2 g, 3 g, 4 g, 5 g, 6 g, 7 g, 8 g, or 9 g of at least one soluble anionic fiber per serving.

A fluid phase may include an alginate soluble anionic fiber and/or a pectin soluble anionic fiber. In certain cases, an alginate soluble anionic fiber and a pectin soluble anionic fiber are used. A fiber blend as described herein can be used to provide the alginate soluble anionic fiber and/or the pectin soluble anionic fiber. An alginate and pectin can be any type and in any form, as described previously. For example, an alginate can be a high, medium, or low molecular weight range alginate, and a pectin can be a high-methoxy pectin. Also as indicated previously, two or more alginate forms can be used, such as a high molecular weight and a low molecular weight alginate, or two high molecular weight alginates, or two low molecular weight alginates, or a low and a medium molecular weight alginate, etc. For example, Manugel GHB alginate and/or Manugel LBA alginate can be used. In other cases, Manugel DPB can be used. Genu Pectin, USPL200 (a high-methoxy pectin) can be used as a pectin. In certain cases, potassium salt forms of an soluble anionic fiber can be used, e.g., to reduce the sodium content of an ingestible composition.

A fluid phase may include alginate and/or pectin in a total amount of about 0.3% to about 5% by weight, or any value therebetween, e.g., about 1.25% to about 1.9%; about 1.4% to about 1.8%; about 1.0% to about 2.2%, about 2.0% to about 4.0%, about 3.0%, about 4.0%, about 2.0%, about 1.5%, or about 1.5% to about 1.7%. Such percentages of total alginate and pectin can yield about 2 g to about 8 g of fiber per 8 oz. serving, e.g., about 3 g, about 4 g, about 5 g, about 6 g, or about 7 g fiber per 8 oz. serving. In other cases, about 4 g to about 8 g of fiber (e.g., about 5 g, about 6 g, or about 7 g) per 12 oz. serving can be targeted. In some embodiments, about 1.7% fiber by weight of a fluid ingestible composition is targeted.

In some cases, a fluid phase may include only alginate as a soluble anionic fiber. In other cases, alginate and pectin are used. A ratio of alginate to pectin (e.g., total alginate to total pectin) in a fluid ingestible composition can range from about 8:1 to about 1:8, and any ratio therebetween (e.g., alginate:pectin can be in a ratio of about 1:1, about 1.2:1, about 1.3:1, about 1.4:1, about 1.5:1, about 1.6:1, about 1.62:1, about 1.7:1, about 1.8:1, about 1.9:1, about 2:1, about 3:1, about 4:1, about 5:1, about 5.3:1, about 5.6:1, about 5.7:1, about 5.8:1, about 5.9:1, about 6:1, about 6.1:1, about 6.5:1, about 7:1, about 7.5:1, about 7.8:1, about 2:3, about 1:4, or about 0.88:1). In cases where alginate and pectin are in a ratio of about 0.5:1 to about 2:1, it is believed that pectin and alginate electrostatically associate with one another to gel in the absence of multivalent cations; thus, while not being bound by theory, it may be useful to delay the introduction of multivalent cations until after such gel formation. In other cases, where the ratio of alginate to pectin is in the range from about 3:1 to about 8:1, it may be useful to include a multivalent cation source such as a calcium source (e.g., to crosslink the excess alginate) to aid gel formation in the stomach. In these cases, the inventors believe, while not being bound by any theory, that the lower amount of pectin protects the alginate from precipitating as alginate at the low pHs of the stomach environment, while the multivalent cation source cross-links and stabilizes the gels formed.

A fluid phase can have a pH from about 3.9 to about 4.5, e.g., about 4.0 to about 4.3 or about 4.1 to about 4.2. At these pHs, it is believed that the fluid phase are above the pKas of the alginate and pectin acidic subunits, minimizing precipitation, separation, and viscosity of the solutions. In some cases, malic, phosphoric, and citric acids can be used to acidify the compositions. In some cases, a fluid ingestible composition can have a pH of from about 5 to about 7.5. Such fluid ingestible compositions can use pH buffers known to those having ordinary skill in the art.

Sweeteners useful in the present invention can vary according to the use of the composition. Low glycemic sweeteners including trehalose, isomaltulose, aspartame, saccharine, and sucralose can be used. Sucralose can be used alone in certain formulations. The choice of sweetener will impact the overall caloric content of a composition. In certain cases, a composition can be targeted to have 40 calories/12 oz serving.

A fluid phase can demonstrate gel strengths of about 20 to about 250 grams force (e.g., about 60 to about 240, about 150 to about 240, about 20 to 30, about 20 to about 55, about 50 to 200; about 100 to 200; and about 175 to 240), as measured in a static gel strength assay. Gel strengths can be measured in the presence and absence of a multivalent cation source, such as a calcium source.

A fluid phase can exhibit a viscosity in the range of from about 15 to about 100 cPs, or any value therebetween, at a shear rate of about 10^{-S}, e.g., about 17 to about 24; about 20 to about 25; about 50 to 100, about 25 to 75, about 20 to 80, or about 15 to about 20 cPs. Viscosity can be measured by those skilled in the art, e.g., by measuring flow curves of solutions with increasing shear rate using a double gap concentric cyclinder fixture (e.g., with a Parr Physica Rheometer).

A fluid phase can include a multivalent cation sequestrant, e.g., to prevent premature gelation of the soluble anionic fibers. A multivalent cation sequestrant can be selected from EDTA and its salts, EGTA and its salts, sodium citrate, sodium hexametaphosphate, sodium acid pyrophosphate, trisodium phosphate anhydrous, tetrasodium pyrophosphate, sodium tripolyphosphate, disodium phosphate, sodium carbonate, and potassium citrate. A multivalent cation sequestrant can be from about 0.001% to about 0.3% by weight of the ingestible composition. Thus, for example, EDTA can be used at about 0.0015%to about 0.002% by weight of the ingestible composition and sodium citrate at about 0.230% to about 0.260% (e.g., 0.250%) by weight of the ingestible composition.

A fluid phase can include a juice or juice concentrate and optional flavorants and/or colorants. Juices for use include fruit juices such as apple, grape, raspberry, blueberry, cherry, pear, orange, melon, plum, lemon, lime, kiwi, passionfruit, blackberry, peach, mango, guava, pineapple, grapefruit, and others known to those skilled in the art. Vegetable juices for use include tomato, spinach, wheatgrass, cucumber, carrot, peppers, beet, and others known to those skilled in the art.

The brix of the juice or juice concentrate can be in the range of from about 15 to about 85 degrees, such as about 25 to about 50 degrees, about 40 to about 50 degrees, about 15 to about 30 degrees, about 65 to about 75 degrees, or about 70 degrees. A fluid ingestible composition can have a final brix of about 2 to about 25 degrees, e.g., about 5, about 10, about 12, about 15, about 20, about 2.5, about 3, about 3.5, about 3.8, about 4, or about 4.5.

Flavorants can be included depending on the desired final flavor, and include flavors such as kiwi, passionfruit, pineapple, coconut, lime, creamy shake, peach, pink grapefruit, peach grapefruit, pina colada, grape, banana, chocolate, vanilla, cinnamon, apple, orange, lemon, cherry, berry, blueberry, blackberry, apple, strawberry, raspberry, melon(s), coffee, and others, available from David Michael, Givaudan, Duckworth, and other sources.

Colorants can also be included depending on the final color to be achieved, in amounts quantum satis that can be determined by one having ordinary skill in the art.

Solids

At least one soluble anionic fiber can be present in a solid ingestible composition in any form or in any mixtures of forms. A solid phase can be a processed, unprocessed, or both. Processed forms include extruded forms, spray-dried forms, roll-dried forms, or dry-blended forms. For example, a snack bar can include at least anionic soluble anionic fiber present as an extruded food product (e.g., a crispy), at least one soluble anionic fiber in an unextruded form (e.g., as part of the bar), or both.

A formed food product can be cold- or hot-extruded and can assume any type of extruded form, including without limitation, a bar, cookie, bagel, crispy, puff, curl, crunch, ball, flake, square, nugget, and snack chip. In some cases, a formed food product is in bar form, such as a snack bar or granola bar. In some cases, a formed food product is in cookie form. In other cases, a formed food product is in a form such as a crispy, puff, flake, curl, ball, crunch, nugget, chip, square, chip, or nugget. Such formed food products can be eaten as is, e.g., cookies, bars, chips, and crispies (as a breakfast cereal) or can be incorporated into a solid ingestible composition, e.g., crispies incorporated into snack bars.

A solid form may also be a lollipop or a lolly that is made of hardened, flavored sugar mounted on a stick and intended for sucking or licking. One form of lollipop has a soft-chewy filling in the center of the hardened sugar. The soft center filling may be a gum, fudge, toffee, caramel, jam, jelly or any other soft-chewy filling known in the art. The at least one multivalent cation may be in the soft-chewy center or the harnend sugar. Likewise, at least one anionic soluble fiber may be in the soft-chewy center or the harnend sugar. A hard candy filled with a soft center filling is another embodiment of the present invention. This embodiment is similar to the lollipop, except it is not mounted on a stick. The soft-chewy filling may be in the center or swirled or layered with the hard sugar confection.

A cookie or mini-bar can include at least one soluble anionic fiber in an unformed form or in a formed (e.g., extruded) form. A snack chip can include at least one soluble anionic fiber in extruded form or in spray-dried form, or both, e.g., an extruded soluble anionic fiber-containing chip having at least one anionic soluble fiber spray-dried on the chip.

A solid ingestible composition can include optional additions such as frostings, icings, coatings, toppings, drizzles, chips, chunks, swirls, or layers. Such optional additions can include at least one multivalent cation, at least one soluble anionic fiber, or both.

Solid ingestible compositions can provide any amount from about 0.5 g to about 10 g total soluble anionic fiber per serving, e.g., about 0.5 g to about 5 g, about 1 g to about 6 g, about 3 g to about 7 g, about 5 g to about 9 g, or about 4 g to about 6 g. For example, in some cases, about 1 g, about 2 g, about 3 g, about 4 g, about 5 g, about 6 g, about 7 g, about 8 g, or about 9 g of soluble anionic fiber per serving can be provided.

A solid ingestible composition can include at least one soluble anionic fiber at a total weight percent of the ingestible composition of from about 4% to about 50% or any value therebetween. For example, a solid ingestible composition can include at least one soluble anionic fiber of from about 4% to about 10% by weight; or about 5% to about 15% by weight; or about 10% to about 20% by weight; or about 20% to about 30% by weight; or about 30% to about 40% by weight; or about 40% to about 50% by weight.

An extruded food product can be from about 0% to 100% by weight of an ingestible composition, or any value therebetween (about 1% to about 5%; about 5% to about 10%; about 10% to about 20%; about 20% to about 40%; about 30% to about 42%; about 35% to about 41%; about 37% to about 42%; about 42% to about 46%; about 30% to about 35%; about 40% to about 50%; about 50% to about 60%; about 60% to about 70%; about 70% to about 80%; about 80% to about 90%; about 90% to about 95%; about 98%; or about 99%). For example, an extruded bar, cookie, cracker, or chip can be about 80% to about 100% by weight of an ingestible composition or any value therebetween.

Alternatively, an ingestible composition can include about 30% to about 55% by weight of an extruded food product or any value therebetween, e.g., about 32%, about 33%, about 34%, about 35%, about 36%, about 37%, about 38%, about 39%, about 40%, about 42%, about 45%, about 48%, about 50%, about 52%, or about 54% by weight of an extruded food product. For example, a snack bar composition can include extruded crispies in an amount of from about 32% to about 46% by weight of the snack bar.

An ingestible composition or extruded food product can include one or more of the following: cocoa, including flavonols, and oils derived from animal or vegetable sources, e.g., soybean oil, canola oil, corn oil, safflower oil, sunflower oil, etc. For example, an extruded food product can include cocoa or oils in an amount of about 3% to about 10% (e.g., about 3% to about 6%, about 4% to about 6%, about 5%, about 6%, about 7%, or about 4% to about 8%) by weight of the extruded food product.

One embodiment of the present invention is a stable two phase product having at least one soluble anionic fiber and at least one multivalent cation in the same product, but formulated so that the soluble anionic fiber and multivalent cation do not react during processing or prior to ingestion, but react following ingestion as a standard multivalent cation-anion fiber reaction. One product design includes a fluid phase center and a crisp solid phase outside the fluid phase. One embodiment places the soluble anionic fiber in the fluid phase and places the multivalent cation in the solid phase, e.g., alginate and pectin in the jam and calcium fumarate in the baked dough. This embodiment, while advantageous over more homogeneous highly gel forming compositions is less than optimal from an organoleptic standpoint. Specifically it provided a somewhat rubber-like jam phase instead of pleasant texture.

Another embodiment of the present invention addresses this issue, wherein this problem is solved by adding the soluble anionic fiber to the baked dough solid phase and the multivalent cation to the fluid jam phase. Such an embodiment provides a cookie that reduces the water activity of the fiber-containing phase, which restricts the fiber so that it is prevented from reacting with the multivalent cation in the jam. The placement of the multivalent cation into a postbake, medium water activity filler, e.g., the fluid phase, allows the cation to be formulated in the product with an acceptable organoleptic profile and an inability to react with fiber even if minor migration occurs.

The water activity of both components can be further adjusted to deliver a product with not only restrictive reaction in place but acceptable eating qualities and the right characteristics needed to for ease of manufacturing.

Types of salts useful include calcium fumarate, tricalcium phosphate, dicalcium phosphate dihydrate and calcium carbonate. The gram weight tested will vary depending on the salt type due to its characteristic calcium load. The piece weight of the product under discussion has been about 13 to about 20 g, with each piece delivering about 50 to about 75 kcal.

BENEFAT® is a family of triglyceride blends made from the short and long chain fatty acids commonly present in the diet. It is the uniqueness of these fatty acids that contribute to the range's reduced calorie claim. BENEFAT® products are designed to replace conventional fats and oils in dairy, confectionery and bakery products, giving full functionality with significantly reduced energy and fat content. BENEFAT® is the Danisco trade name for SALATRIM, the abbreviation for short and long-chain triglyceride molecules. The short-chain acids (C₂-C₄) may be acetic, propionic, butyric or a combination of all three, while the long-chain fatty acid (C₁₆-C₂₂) is predominantly stearic and derived from fully hardened vegetable oil. Unlike other saturated fatty acids, stearic acid has a neutral effect on blood cholesterol. BENEFAT® is also free of trans fatty acids and highly resistant to oxidation. Compared to the 9 calories per gram of traditional fat, BENEFAT® contains just 5 calories per gram (US regulation) or 6 calories per gram (EU regulation), at the same time giving foods a similar creamy taste, texture, and mouthfeel as full-fat products. Metabolism upon consumption occurs in much the same way as with other food components.

Preferred product features include about 500 to about 1500 mg of alginate and the multivalent cation is calcium wherein about 50 to about 500 mg of elemental calcium are delivered. The product has low calories between about 50 to about 100 calories and can be a cookie/mini-bar/cookie/cereal bar with a jam filling.

The ingestible composition of the present invention can be provided in any package, such as enclosed in a wrapper or included in a container. An ingestible composition can be included in an article of manufacture. An article of manufacture that includes an ingestible composition described herein can include auxiliary items such as straws, napkins, labels, packaging, utensils, etc.

An article of manufacture can include a source of at least one multivalent cation. For example, a source of at least one multivalent cation can be provided as a fluid, e.g., as a beverage to be consumed before, during, or after ingestion of the ingestible composition. In other cases, at least one multivalent cation can be provided in a solid or gel form. For example, a source of at least one multivalent cation can be provided in, e.g., a jelly, jam, dip, swirl, filling, or pudding, to be eaten before, during, or after ingestion of the ingestible composition. Thus, in some embodiments, an article of manufacture that includes a cookie or bar solid ingestible composition can also include a dip comprising a source of at least one multivalent cation, e.g., into which to dip the cookie or bar solid ingestible composition.

Also provided are articles of manufacture that include a fluid ingestible composition. For example, a fluid ingestible composition can be provided in a container. Supplementary items such as straws, packaging, labels, etc. can also be included. Alternatively, the soluble anionic fiber may be included in a beverage and the multivalent cation may be provided inside, outside or both of a straw or stirring stick. In some cases, at least one multivalent cation, as described below, can be included in an article of manufacture. For example, an article of manufacture can include a fluid ingestible composition in one container, and a source of multivalent cations in another container. Two or more containers may be attached to one another.

Methods of Weight Management

A soluble anionic fiber (such as alginate and pectin) is orally administered concurrently with a multivalent cation source such as a water-soluble calcium salt, to reduce caloric intake, inducing satiety, reducing weight and/or improving weight reduction. Continued use of these compositions by individuals will result in a cumulative decrease in caloric consumption, which will result in weight loss or diminished weight gain. Although not wishing to be bound by theory, the inventors hypothesize that the multivalent cation calcium ions of the soluble calcium source cross link the carboxylate groups on the fiber molecules, resulting in the formation of highly viscous or gelled materials. This gelling effect increases the viscosity of the gastric and intestinal contents, slowing gastric emptying, and also slowing the rate of macro-nutrient, e.g., glucose, amino acids, fatty acids, and the like, absorption. These physiological effects prolong the period of nutrient absorption after a meal, and therefore prolong the period during which the individual experiences an absence of hunger. The increased viscosity of the gastrointestinal contents, as a result of the slowed nutrient absorption, also causes a distal shift in the location of nutrient absorption. This distal shift in absorption may trigger the so-called “ileal brake”, and the distal shift may also cause in increase in the production of satiety hormones such as GLP-1 and PYY.

Provided herein are methods employing the ingestible compositions described herein. For example, a method of inducing satiety and/or satiation in an animal is provided. The method can include administering an ingestible composition to an animal. An animal can be any animal, including a human, monkey, mouse, rat, snake, cat, dog, pig, cow, sheep, horse, bird, or horse. Administration can include providing the ingestible combination either alone or in combination with other meal items. Administration can include co-administering, either before, after, or during administration of the ingestible composition, a source of at least one multivalent cation, such as calcium or a sequestered source of calcium, as described herein. At least one multivalent cation can be administered within about a four hour time window flanking the administration of the ingestible composition. For example, a source of calcium, such as a solution of calcium lactate, can be administered to an animal immediately after the animal has ingested a fluid ingestible composition as provided herein. Satiety and/or satiation can be evaluated using consumer surveys (e.g., for humans) that can demonstrate a statistically significant measure of increased satiation and/or satiety. Alternatively, data from paired animal sets showing a statistically significant reduction in total caloric intake or food intake in the animals administered the ingestible compositions can be used as a measure of facilitating satiety and/or satiation.

As indicated previously, the ingestible compositions provide herein can hydrate and gel in the stomach and/or small intestine, leading to increased viscosity in the stomach and/or small intestine after ingestion. Accordingly, provided herein are methods for increasing the viscosity of stomach and/or small intestine contents, which include administering an ingestible composition to an animal. An animal can be any animal, as described above, and administration can be as described previously. Viscosity of stomach contents can be measured by any method known to those having ordinary skill in the art, including endoscopic techniques, imaging techniques (e.g., MRI), or in vivo or ex vivo viscosity measurements in e.g., control and treated animals.

Also provided are methods for promoting weight loss by administering an ingestible composition as provided herein to an animal. Administration can be as described previously. The amount and duration of such administration will depend on the individual's weight loss needs and health status, and can be evaluated by those having ordinary skill in the art. The animal's weight loss can be measured over time to determine if weight loss is occurring. Weight loss can be compared to a control animal not administered the ingestible composition.

Provided here are methods for improving weight reduction by at least 5% in an animal by orally administering to the animal a serving of a formed food having at least one soluble anionic fiber and a multivalent cation, wherein the weight reduction improvement is measured after four months of daily administration of the ingestible composition. The improvement in weight reduction ican be at least about 10%, at least about 15%, and at least about 20%.

Methods of Diabetes Management

A soluble anionic fiber (such as alginate and pectin) is orally administered concurrently with a multivalent cation source such as a water-soluble or relatively water-insoluble calcium or other multivalent cation salt in close proximity to the consumption of a meal to reduce the elevations in blood glucose and insulin that accompany the consumption of a meal, particularly if the meal contains a considerable amount of glucose, starch, or other carbohydrates. Concurrent administration means that the compositions are ingested as part of the meal, or they are ingested before the meal, preferably about one to 90 minutes before a meal, and more preferably about one to 60 minutes before a meal, or they are ingested after the completion of a meal, preferably about one to 90 minutes after completion of a meal, and more preferably about one to 60 minutes after completion of a meal. Water-soluble calcium salts include such salts as fumarate, citrate, or lactate. Relatively water-insoluble calcium salts include the phosphate salts. The product may be administered in any comestible form, including a bar, a cookie, tablets, baked goods or beverages. Although not being bound by theory, the inventors believe that the increased viscosity of the gastric and intestinal contents resulting from the consumption of the anionic fiber and calcium compositions slows the absorption of glucose present in, or derived from, a meal consumed concurrently with the ingestion of the comestible form containing anionic fiber and a multivalent cation. The slowed absorption of glucose mitigates the increase in blood glucose levels that accompany the consumption of such a meal. The decreased glucose levels in the blood decreases the demands on the pancreas to release insulin into the circulation, and blood insulin levels are also decreased.

The value of decreasing postprandial glucose and insulin levels in the treatment of diabetes is well recognized. According to the “Position of the American Dietetic Association: Health implications of dietary fiber” (Journal of the American Dietetic Association, 102(7):993-1000 (2002), which if fully incorporated herein by reference), “[c]onsiderable experimental evidence demonstrates that the addition of viscous dietary fibers slows gastric emptying rates, digestion, and the absorption of glucose to benefit immediate postprandial glucose metabolism and long-term glucose control in individuals with diabetes mellitus”. Clinical studies have demonstrated the beneficial effects of soluble fiber in decreasing postprandial glucose and insulin levels, and have further recognized the significance of these reductions in treating and controlling the progression of type 2 diabetes. For example, Chandalia et al (Beneficial effects of high dietary fiber intake in patients with type 2 diabetes mellitus, The New England Journal of Medicine 343:1392-1398 (2000), with is incorporated fully herein by reference) tested in a crossover trial the effects of a high fiber diet in subjects with diagnosed type 2 diabetes. The high fiber diet decreased both postprandial glucose and insulin levels. The authors concluded “an increase in the intake of dietary fiber, predominantly of the soluble type, by patients with type 2 diabetes mellitus improved glycemic control and decreased hyperinsulinemia in addition to the expected lowering of plasma lipid concentrations” (p. 1397). While not wishing to be bound by theory, the inventors believe that the decreased blood glucose levels resulting from the consumption of the compositions of the present invention with meals decreases demand on the beta cells of the pancreas to release insulin. Pancreatic insulin demand is therefore the need for the pancreas, in responding to glucose or carbohydrate intake, to release insulin in order to maintain stable glucose levels. The decreased demand on the beta cells helps to prolong their function of being able to release sufficient levels of insulin, and this prolonged function helps to reduce the severity of diabetes and slow the progression of the disease. Furthermore, it is contemplated that the continued consumption of the compositions of the present invention in conjunction with, or as an accompaniment to, meals will improve the treatment of diabetes, as measured by decreases in the blood hemoglobin Alc levels (a biochemical marker for the long-term presence in the blood of elevated glucose levels). Additionally, such continued consumption of these compositions by individuals at risk of diabetes, such as individuals with the metabolic syndrome, or individuals with impaired glucose tolerance, will reduce the risk of these individuals advancing to diabetes as a result of the lowered glucose levels and the reduced demand on the pancreas. By continued use is meant the consumption of these compositions in conjunction with one, two, or three meals per day, preferably two or three meals per day, and most preferably three meals per day, for a period from about seven days to several decades, more preferably about 30 days to several decades, and most preferably about 60 days to several decades. Importantly, the benefit achieved is proportional to the continued use of the product over time, the magnitude of the benefit will be related to the cumulative ingestion of the inventive compositions.

In one aspect of the invention, compositions are intended as accompaniment to a normal meal, in comparison to a meal replacement product, typically a canned or bottled shelf stable beverage, or a bar, that is consumed in place of a normal meal. By “normal meal” is meant the morning, mid-day, or evening food consumption that is characteristic of the western diet, and frequently consists of a combination of vegetables, starches, and a protein source, either served individually or combined in a single consumable form.

The inventors have determined that a fiber containing composition, when consumed concurrently with a meal, reduces the blood glucose and insulin responses to the meal. Importantly, this is distinguished from previous inventions related to meal replacements or substitutes. Meal replacements often contain a substantial calorie load, generally about 200 to 250 kcal. These meal replacement products are ill-suited for the objectives of the current invention, since their relatively high caloric load would substantially increase the caloric and glycemic load if they were ingested in conjunction with a meal. In contrast, the compositions of the current composition contain about 50 to 100 kcal. The present invention provides a blood glucose lowering effective amount or a blood insulin lowering effective amount of a soluble fiber that is consumed with a meal in a separate comestible form; i.e. consumed as a meal adjuvant as opposed to a meal replacement. The soluble fiber is preferably an anionic fiber, more preferably alginic acid, which is consumed with a multivalent cation source, particularly a calcium source. Additionally, the embodiments of the current invention are distinguished from the consumption of a meal or diet supplemented with soluble fiber. This may consist of individual foods with a high level of soluble fiber, or the addition of soluble fiber to a single food meal such as casserole. A preferred embodiment of the present invention is therefore a comestible containing soluble fiber that is consumed concurrently with a meal.

The inventors contemplate that these compositions will be consumed with a meal. Also contemplated is the consumption of these compositions prior to a meal. Such consumption before a meal may occur from about one minute to about 90 minutes before a meal. Further contemplated is the consumption of these compositions after a meal. Such consumption after a meal may occur from about one minute to about 90 minutes after meal. Without being bound by theory, the inventors believe that the reservoir function of the stomach, along with the relative slow postprandial gastric emptying, will permit the soluble anionic fiber and the multivalent cation of the inventive compositions to mix with the stomach contents, thereby increasing the viscosity of the contents and slowing glucose absorption.

Elevated postprandial glucose and insulin levels contribute to the development or progression of type 2 diabetes, and mitigation of this elevation will slow the development or progression of this disease. Therefore methods are also provided for preventing or ameliorating the development of diabetes, slowing the progression of diabetes, and treating diabetes. Administration can be as described previously. The amount and duration of such administration will depend on the individual's stage of developing diabetes, and can be determined by those with ordinary skill in the art.

The following examples are representative of the invention, and are not intended to be limiting to the scope of the invention.

### EXAMPLES

### Example 1

A cookie having a solid phase, e.g., a baked dough phase, containing a soluble anionic fiber blend and a fluid phase, e.g., jam phase containing a soluble calcium source deposited in the baked dough phase was produced.

The baked dough phase was prepared by adding BENEFAT® and lecithin to a premix of flour, cellulose, egg white, salt, leavening and flavors in a Hobart mixer and creaming by mixing at low speed for about 1 minute followed by high speed for about 2 minutes. The liquids were added to creamed mixture and blended at medium speed for about 2 minutes.

The fiber blend used contained about 46% sodium alginate LBA (ISP, San Diego, Calif.), about 39.6% sodium alginate GHB (ISP), and about 14.4% pectin (USP-L200, Kelco, San Diego, Calif.).

The fiber blend and glycerin were added to a separate bowl and combined. This combined fiber/glycerin material was added to the other ingredients in the Hobart mixer and was mixed on medium speed for about 1 minute. The resulting dough was then sheeted to desired thickness on a Rhondo sheeter and a dough pad measuring about 3 inched by about 6 inches was created.

The jam phase was prepared by adding a premixed BENEFAT® calcium source mixture to the jam base and mixed until uniformly mixed. A predetermined amount of the jam was then added onto the top surface of the cookie dough pad. The dough pad edges were wetted and sealed. Bars were baked at 325° F. for about 9 minutes, cut, cooled and the resulting cookies were individually packaged. The total caloric value of each cookie was about 50 kcal.

| | % Dough | % Total |
|---|---|---|
| Ingredient | Phase | Formulation |
| Flour all purpose | 29.140 | 12.165 |
| Cellulose, solka floc - | 6.980 | 2.914 |
| International Fiber Corp. | | |
| Powder egg white | 0.580 | 0.242 |
| Salt (NaCl) | 0.200 | 0.083 |
| Sodium Bicarbonate Grade #1 | 0.510 | 0.213 |
| Cookie Dough Flavor | 0.170 | 0.071 |
| BENEFAT | 2.060 | 0.860 |
| Lecithin | 0.640 | 0.267 |
| Polydextrose Litesse 70% syrup, | 15.870 | 6.625 |
| Ultra | | |
| Water | 11.830 | 4.939 |
| Liquid Vanilla flavor | 0.280 | 0.117 |
| sucralose, 25% liquid. | 0.090 | 0.038 |
| Potassium sorbate | 0.250 | 0.104 |
| Alginate fiber blend | 17.400 | 7.264 |
| Glycerine, Optim 99.7% USP | 14.000 | 5.845 |
| | 100.000 | 41.70 |

| | % Fluid | % Total |
|---|---|---|
| Ingredient | Phase | Formulation |
| BENEFAT | 21.100 | 12.291 |
| Calcium Fumarate Trihydrate | 11.000 | 6.408 |
| Reduced Calorie Strawberry | 67.900 | 39.553 |
| Filling (Smuckers) | | |
| | 100.000 | 58.25 |

Control

Solid Phase:

| | % Dough | % Total |
|---|---|---|
| Ingredient | Phase | Formulation |
| Flour - all purpose | 29.140 | 12.530 |
| Cellulose, solka floc - | 6.980 | 3.001 |
| International Fiber Corp. | | |
| Powder egg white | 0.580 | 0.249 |
| Salt (NaCl) | 0.200 | 0.086 |
| Sodium bicarbonate Grade #1 | 0.510 | 0.219 |
| Cookie dough flavor | 0.170 | 0.073 |
| BENEFAT | 19.450 | 8.364 |
| Lecithin | 0.640 | 0.275 |
| Polydextrose Litesse 70% syrup, | 15.870 | 6.824 |
| Ultra | | |
| Water | 11.830 | 5.087 |
| Liquid vanilla flavor | 0.280 | 0.120 |
| sucralose, 25% liquid. | 0.090 | 0.039 |
| Potassium sorbate | 0.250 | 0.108 |
| Alginate fiber blend | 0.000 | 0.000 |
| Glycerine, Optim 99.7% USP | 14.000 | 6.020 |
| | 100.000 | 43.00 |

| | | % Total |
|---|---|---|
| Ingredient | % Fluid Phase | Formulations |
| BENEFAT | 32.100 | 19.260 |
| Reduced Calorie | 67.900 | 40.740 |
| Strawberry Filling | | |
| (SMUCKERS) | | |
| Total | 100.000 | 60.00 |

Intestinal Viscosity Measurement

Fully grown female Yucatan minipigs (Charles River Laboratories, Wilmington, Mass.), weighing about 90 kg, were fitted with indwelling silicone rubber sample ports (Omni Technologies, Inc., Greendale, Ind.) implanted in a surgically created dermal fistula at the ileocecal junction. The sample ports were sealed by a removable cap. These ports permit removal of samples of digesta as it passed from the ileum to the cecum. Additional details of this procedure are presented in B. Greenwood van-Meerveld et al., Comparison of Effects on Colonic Motility and Stool Characteristics Associated with Feeding Olestra and Wheat Bran to Ambulatory Mini-Pigs, Digestive Diseases and Sciences 44:1282-7 (1999), which is fully incorporated herein by reference.

Three Yucatan minipigs with the fistulas described above were housed in individual stainless steel pens in a windowless room maintained on a cycle of 12 hours of light and 12 hours of dark. They were conditioned to consume low fiber chow (Laboratory Mini-Pig Diet 5L80, PMI Nutritional International, Brentwood, Mo.). This chow contains about 5.3% fiber. The pigs were fed once each day, in the morning. Water was provided ad lib throughout the day.

Samples are taken from the ileal sample port immediately after feeding, and then at about 30 minute intervals for about 300 minutes. The volume of sample collected is about 50 to 130 ml. All samples are assayed for viscosity within 30 minutes after collection.

Samples of digesta were collected in sealed plastic containers. Viscosity of the digesta were measured with a Stevens QTS Texture Analyzer (Brookfield Engineering, Inc., Middleboro, Mass.). This instrument measures the relative viscosity of digesta by a back extrusion technique. The instrument included a stage plate, a 60 cm vertical tower, a mobile beam and a beam head that contained a load-cell. During back extrusion, the beam descended at a constant rate, and the force required to back extrude the sample was recorded over time. The sample containers were 5 cm deep spherical aluminum cups with an internal diameter of about 2.0 cm. The volume of the cup was about 20 ml. The spherical probe includes a 1.9 cm TEFLON brand ball mounted on a 2 mm threaded rod which is attached to the mobile beam. The diameters of the sample cup and probe allow for a wide range of viscosity (liquid to solid digesta) to be measured without approaching the maximum capacity of the rheometer (25 kg/peak force). During each test, the beam thrusts the probe into the test sample at a constant rate (12 cm/second) for a 2 cm stroke, forcing the sample to back-extrude around the equatorial region of the probe. The peak force for back extrusion at a controlled stroke rate is proportional to the viscosity of the sample. At each time point, 2-6 samples from each pig were tested, and the mean peak force WAS calculated and recorded.

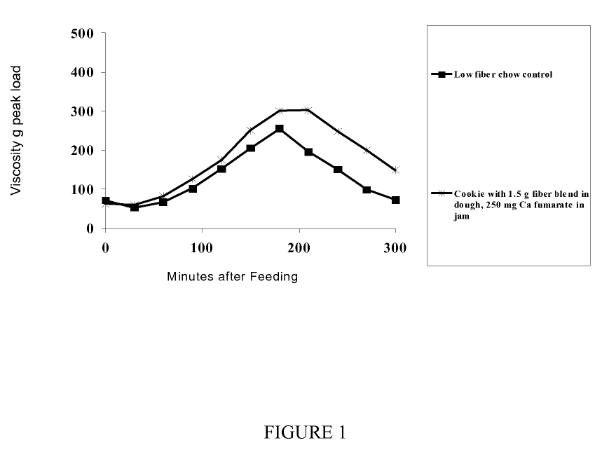

The test for effects of fiber containing cookies on viscosity was performed by providing each pig with its daily ration of low fiber chow (1400 g). Before feeding, one cookie was gently broken into four to six pieces and mixed into the chow. The animals have unlimited access to water during and after feeding. The effects of the cookie of this example containing fiber and calcium on intestinal viscosity is shown in FIG. 1. Each treatment was provided to each of three pigs on three separate days to yield nine replicates for each sample. Each point plotted in FIG. 1 is the mean of these nine determinations. The fiber and calcium-containing cookie produced viscosities significantly greater than those produced by control chow (p<0.05, as measured by a two-tailed t-test) at the time points from 210 minutes through 300 minutes.

### Example 2

Products with the following compositions were prepared by a process similar to that described in Example 1. The water, lecithin, molasses, sucralose, and vanilla were mixed. Benefat and sugar were added, and the mixture was blended on low speed for 20 seconds, and then mixed on high speed for two minutes. The flour, oats, and baking soda were added, and blended on medium for 20 seconds, followed by 10 two minutes at high speed. The fiber blend and glycerin were then added, and mixed on high speed for 30 seconds. Cookies were then formed and baked, as described in

### Example 1

Active

| | | % Dough | % Total |
|---|---|---|---|
| Ingredient | | Phase | Formulation |
| Benefat | Danisco | 18.200 | 10.69 |
| Sugar | Market | 18.200 | 10.69 |
| Water | Tap | 2.900 | 1.704 |
| Molasses, Black Strap #150 | Sweetener | 2.400 | 1.410 |
| | Supply | | |
| Sucralose, 25% Solution | Tate & Lyle | 0.034 | 0.020 |
| Lecithin | ADM | 0.750 | 0.441 |
| Vanilla, N&A 597970 T | Firminich | 0.800 | 0.470 |
| Flour, Cookie | Minell | 15.200 | 8.930 |
| | Milling | | |
| Baking Soda | Arm & | 0.250 | 0.147 |
| | Hammer | | |
| Quick Rolled Oats | Can-Oat | 21.766 | 12.788 |
| | Milling | | |
| Fiber Blend* | | 16.000 | 9.400 |
| Glycerine, Optim 99.7% USP | Procter & | 3.500 | 2.056 |
| | Gamble | | |
| | | 100.000 | 41.70 |

Fiber blend was prepared by mixing about 46% sodium alginate LBA (ISP, San Diego, Calif.), about 39.6% sodium alginate GHB (ISP), and about 14.4% pectin (USP-L200, Kelco, San Diego, Calif.), and agglomerating the mix. Each cookie contained 1.5 g of fiber derived from alginate and pectin. The calorie content of each cookie was about 50 kilocalories.

Placebo

These cookies were prepared as described for the fiber-containing cookies above, except for the deletion of the fiber blend and rebalancing of the other ingredients. The placebo differed from the alginate-containing sample only in that it lacked the fiber blend and contained a compensatory mass of wheat flour. Therefore, the placebo cookie contained an additional 1.5 g of wheat flour, and a serving of two placebo cookies contained an additional 3 g. Using the commonly accepted value of wheat flour containing about 88 kcal per ounce, this additional flour would have contributed an additional 9-10 kcal per serving of two. However, alginate and pectin are fermentable fibers, and the fermentation products result in these fibers also contributing a caloric load. Therefore, the actual difference in caloric content is less that 9-10 kcal. For the purposes of this invention, the inventors intend that the term “equicaloric” means a difference in caloric content of two compositions of about 10% or less.

| | | % Dough | % Total |
|---|---|---|---|
| Ingredient | | Phase | Formulation |
| Benefat | Danisco | 18.200 | 10.69 |
| Sugar | Market | 18.200 | 10.69 |
| Water | Tap | 4.300 | 2.526 |
| Molasses, Black Strap #150 | Sweetner | 2.400 | 1.410 |
| | Supply | | |
| Sucralose, 25% Solution | Tate & Lyle | 0.034 | 0.020 |
| Lecithin | ADM | 0.750 | 0.441 |
| Vanilla, N&A 597970 T | Firminich | 0.800 | 0.470 |
| Flour, Cookie | Minnel | 29.800 | 17.508 |
| | Milling | | |
| Baking Soda | Arm & | 0.250 | 0.147 |
| | Hammer | | |
| Quick Rolled Oats | Can-Oat | 21.766 | 12.788 |
| | Milling | | |
| Glycerine, Optim 99.7% USP | Procter & | 3.500 | 2.056 |
| | Gamble | | |
| | | 100.000 | 41.70 |

All cookies were topped with a calcium containing jam prepared as follows. Strawberry jam (DeGussa 1111590, 88%) was placed in the bowl of a mixer. The jam as stirred at low speed, and calcium fumarate trihydrate (Sol-U-Cal, Bartek, 12%) was added. The sides of the bowl were scraped frequently to ensure uniform mixing. Mixing was continued until the calcium fumarate was thoroughly dispersed in the jam. The strawberry jam was applied to the surface of the baked cookies.

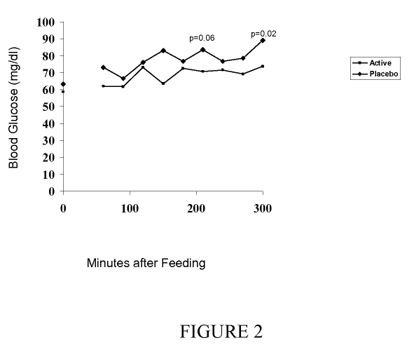

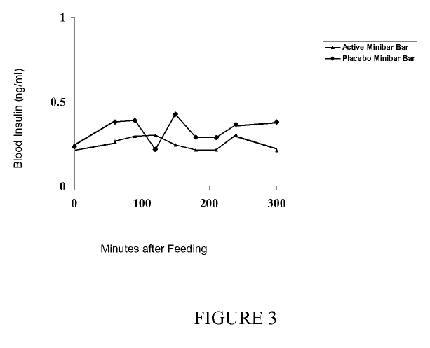

A fully grown female Yucatan minipig (Charles River Laboratories, Wilmington, MA), weighing about 90 kg, was fitted with an indwelling silicone rubber sample port (Omni Technologies, Inc., Greendale, IN) implanted in a surgically created dermal fistula at the ileocecal junction, as described above. The test animal was also fitted with a titanium vascular access port (VAP, Instech Solomon, 5209 Militia Hill Rd, Plymouth Meeting, Pa.) to allow frequent blood sampling without undue stress on the animal, and without physical restraint of the animal. The VAP was surgically implanted into the jugular vein, and a subcutaneous tunnel to the dorsal midline was created. The VAP was located in the tunnel, with the external access portion of the VAP located in a dorsal position near the midline that allowed ready access and minimized disturbance of the site by the animal. Details of the VAP and its implantation have been published elsewhere (Swindle, MM, Vascular access port (VAP) usage in large animal species, Contemporary Topics 44:7-17 (2005)) and are incorporated herein by reference. Blood samples were collected aseptically at the indicated time intervals from a catheter inserted in the VAP. Blood glucose measurements were performed with a Beckman Glucose Analyzer (glucose oxidase assay). An enzyme-linked immunosorbent assay (ELISA) kit (Alpco Diagnostics, Windham, Mass.) specific for porcine insulin was used to determine insulin levels; all insulin assays were performed in duplicate. On test days, the animal was fed a meal of 1000 grams of chow (Laboratory Mini-Pig Diet 5L80) at approximately 7 AM. The animal was fasted for 24 hours prior to feeding. Depending on the treatment administered, two of the cookies of either the fiber or control compositions (as described in this example) were broken into pieces and mixed into the chow. Both the fiber and control compositions were tested on duplicate days. Ad libitum access to water was provided throughout the experiment. FIG. 2 presents the mean blood glucose levels over time for the two treatments. FIG. 3 similarly presents the blood insulin levels. Ingestion of the fiber product resulted in a decrease in blood glucose and insulin levels, in comparison to blood glucose and insulin levels for the control composition. The area under the curve (AUC) was calculated for insulin levels over the interval of 60 minutes to 300 minutes by summing the trapezoidal areas of the plotted intervals. The AUC for insulin after alginate fiber consumption was reduced by 23%, in comparison to the control sample. The AUC was similarly calculated for glucose levels over the interval of 60 to 300 minutes. The glucose AUC observed with the administration of the composition containing alginate was reduced by 11.6%, in comparison to the control composition. Thus the fiber composition administered with the meal of chow was able to reduce the test animal's glucose and insulin response to that meal.

A crisp bar containing high molecular weight alginate and a placebo bar were also tested, following the procedure described above.

| | Ingredients | % |
|---|---|---|
| 1 | Rice Flour (PGP International) | 56.50 |
| 2 | Alginate DPB (ISP) | 31.50 |
| 3 | Whey Protein Isolate BiPro (Davisco) | 4.00 |
| 4 | Corn Starch (Cargill) | 3.00 |
| 5 | Fractionated Canola Oil (Cargill Solo 1000) | 5.00 |
| | Total | 100.00 |

Placebo Crisps:

| | Ingredients | % |
|---|---|---|
| 1 | Rice Flour (PGP International) | 88.00 |
| 2 | Whey Protein Isolate BiPro (Davisco) | 4.00 |
| 3 | Corn Starch (Cargill) | 3.00 |
| 4 | Fractionated Canola Oil (Cargill Solo 1000) | 5.00 |
| | Total | 100.00 |

To produce a batch of crisps, the ingredients were dry blended in a small ribbon blender. The resulting dry blend was transferred using a feeder, e.g., a K-Tron loss-in-weight feeder, into the hopper of an extruder e.g., a Buhler Twin Screw Extruder configured with at least one heating unit, e.g., two Mokon barrel-heating units. Water was added as steam to the dry blend using a two-barrel injection system for adding water and a second liquid into the barrel at variable rates. The blend was then mixed and cooked in the extruder. The hot pressurized product stream was forced through a die for expansion and then conveyed by vacuum to a fluid bed drier, e.g., Buhler fluid bed drier, and dried to the desired moisture content. The fluid bed drier could dry about 50 to about 100 kg/hour at temperatures from about 20° C. to about 11° C.

The crisp bar with alginate and the placebo were prepared as described below.

The crisp fiber bar was an unbaked, formed bar made from formed crisps to provide 3 grams of alginate (Manugel DPB) per bar, that were agglomerated with rolled oats using a syrup containing calcium phosphate (300 mg elemental calcium), and formed into bars. The placebo bar contained similar crisps without alginate. Each 30-g bar contained 100 kcal. Placebo bars were matched for taste, texture, calcium and caloric content, but contained no alginate. The compositions of both of the bars were shown in the following tables. The calories content of the alginate containing bars was about 100 kilocalories.

Alginate Containing Bars:

Containing Fiber Crisps:

| | | | | % in |
|---|---|---|---|---|
| # | Ingredient | Supplier | | Bar |
| | | | % Syrup | |
| 1 | High Maltose Corn Syrup | Cargill | 58.32 | 19.83 |
| 2 | High Fructose Corn Syrup | Cargill | 7.96 | 2.71 |
| 3 | Molasses | Christian Hansen | 3.15 | 1.07 |

Step 1: Weigh and cook above liquid at 160 F.

| | | | | |
|---|---|---|---|---|
| 4 | Fructose | Tate & Lyle | 11.03 | 3.75 |
| 5 | Dicalcium Phosphate | Innophos | 10.50 | 3.57 |
| | Anhydrous | | | |
| 6 | Citric Acid | Cargill | 0.21 | 0.07 |
| 7 | Sugar | United Sugar | 4.73 | 1.61 |

Step 2: Add all dry ingredients, cook to Brix 88%

| | | | | |
|---|---|---|---|---|
| 8 | Canola Oil | Cargill | 2.10 | 0.71 |
| 9 | Vanilla Flavor | Firminich | 2.00 | 0.68 |

Step 3: Add flavors and oil, mix well and cook gently, check Brix to 87%

| | | | | |
|---|---|---|---|---|
| | | | % in Bar | |
| 10 | Active Crisps (see below) | | 33.10 | 33.10 |
| 11 | Rolled Oats | Can-Oat Milling | 18.00 | 18.00 |
| 12 | Raisins, Fresh Whole | Van Drunen | 4.00 | 4.00 |
| 13 | Cranberry Halves | Graceland | 10.90 | 10.90 |

Step 4: Add 680 g syrup to 1320 g dry ingredients, mix quickly
Step 5: Transfer mass to a pan, roll flat, cool at room temperature
for 30 minutes
Step 6: Cut into portion 4 inches long, 2 inches wide, and ½ inch thick
Step 7: Trim to 29 g (plus or minus 1 g) and seal in foil pouch

| | |
|---|---|
| Total | 100 |

Bars Containing Placebo Crisps:

Bars containing placebo crisps were prepared exactly as described for the preparation of bars with fiber crisps, except that placebo crisps were substituted in place of the fiber crisps. The placebo bars contained 3 g of additional rice four, as compared to the alginate-containing bars. As discussed previously in this example, the two compositions differed by only about 9-10 kcal or less in total caloric content, and for the purposes of this discussion they are considered to be equicaloric.

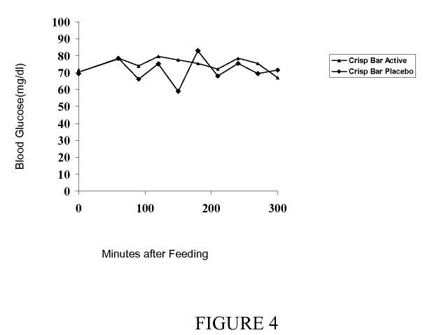

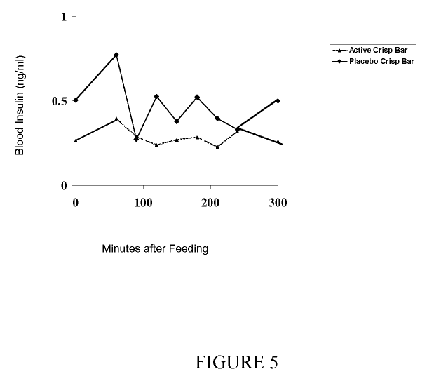

Results for blood glucose and insulin levels are presented in FIG. 4 and FIG. 5. The ingestion of the bar with high molecular weight alginate reduced blood insulin levels, in comparison to the placebo bar treatment. The fiber composition administered with the meal of chow was able to reduce the test animal's insulin response to that meal. The area under the curve (AUC) was calculated for insulin levels over the interval of 60 minutes to 300 minutes by summing the trapezoidal areas of the plotted intervals. The AUC for insulin after alginate fiber consumption was reduced by 35%, in comparison to the control sample. The AUC was similarly calculated for glucose levels over the interval of 60 to 300 minutes. The glucose AUC observed with the administration of the composition containing alginate was reduced by 5.7%, in comparison to the control composition. Insulin is secreted in response to the absorption of glucose in the small intestine. A decreased rate of glucose absorption, as occurs in the presence of soluble viscous fiber in the small intestine, places a lower demand for insulin release from the pancreas, and the circulating levels of insulin are correspondingly decreased. Without being bound by theory, it is believed that the observed decreases in blood insulin levels result from the enhanced intestinal viscosity produced by the combination of alginate and calcium.

### Example 3

Fiber Cookie

The extruded fiber blend of this example was prepared as follows. Hydrocolloids and inulin were dry blended in a small ribbon blender. The resulting dry blend was transferred to a K-Tron loss-in-weight feeder, into the hopper of a Buhler Twin Screw Extruder. Water was injected into the barrel in the approximate ratio of 2 parts water to 2.5-3 parts of dry blend. The dough was mixed and conveyed with the twin screws at an approximate speed of 80-150 RPM at a temperature of 30 to 60° C. With some samples, a second hydrocolloid in solution was injected through a second port. The mixture was then forced through a small die at the end of the screw conveyor and through a cutter, which cut the dough into small pieces. The wet pieces were transferred either manually or pneumatically conveyed to a Buhler fluidized bed drier at 100° C. for approximately 5-20 minutes, depending on the size of the pieces. Two dies were typically used. The first die was approximately 5 mm in diameter, and when the dough was forced through the aperture, small sickle shaped pieces were formed by the cutter. Dimensions when dry were approximately 4-5 mm×8-12 mm×1.2-1.5 mm. The larger pieces were subsequently ground using a lab scale Perten Instruments laboratory disc type mill. The ground material was then sieved to remove fines, and fractions were combined to obtain an approximate 20-40 mesh product. The second die was a small circular die with a 1 mm aperture. This die created very small cylindrical pieces approximately 0.8 mm×1.5 mm when dried. The smaller die eliminated the need for further grinding and sieving.

A cookie base was prepared with the following composition:

| | | | % Final | |
|---|---|---|---|---|
| Ingredient | Supplier | % Dough | Formulation | Amount |
| Benefat | Danisco | 10.250 | 6.022 | 41.00 |
| Corn Oil | Mazola | 3.400 | 1.998 | 13.60 |
| Vitamin E | Eastman | 0.0036 | 0.002 | 0.01 |
| Sugar (Fine | MI Sugar | 14.200 | 8.343 | 56.80 |
| Granulated) | | | | |
| Inulin F97 | Cargill | 2.500 | 1.469 | 10.00 |
| Water | Domestic | 7.700 | 4.524 | 30.80 |
| Molasses, Black | International | 2.400 | 1.410 | 9.60 |
| Strap | Sweeteners | | | |
| Sucralose, 25% | Tate and Lyle | 0.063 | 0.037 | 0.25 |
| Solution | | | | |
| Lecithin Yelkin TM | ADM | 0.750 | 0.441 | 3.00 |
| Vanilla N&A | Firminich | 0.800 | 0.470 | 3.20 |
| 597970 T | | | | |
| Flour, Cookie | Minnel | 15.500 | 9.106 | 62.00 |
| | Milling | | | |
| Baking Soda | Church & | 0.250 | 0.147 | 1.00 |
| | Dwight | | | |
| Quick Rolled Oats | Can-Oat | 17.410 | 10.228 | 69.64 |
| | Milling | | | |
| Fiber Blend (LBA | | 21.280 | 12.502 | 85.12 |
| and GHB, USP | | | | |
| pectin), extruded | | | | |
| with 25% inulin | | | | |
| (Cargill) | | | | |
| Glycerol, Superol | P&G | 3.500 | 2.056 | 14.00 |
| 99.7% | | | | |
| Total | | 100.00 | 58.75 | 400.00 |

The first ten ingredients were combined in a bowl and stirred on low speed in a Kitchen Aid Mixer for 20 seconds. The mix was then stirred on high (setting 5) for two minutes. The flour, baking soda, and quick rolled oats were then added, and the mix was stirred (setting 3) for two additional minutes. The fiber blend and glycerin were then added, the mix was stirred (setting 3) for two more minutes. Walls of the bowl were scraped periodically during mixing. This yielded the final cookie dough. Cookies were formed by weighing 9.4 g of the dough and spreading the dough inside a disk with an internal diameter of 5.1 cm. The dough circles were placed on parchment paper and baked at 177 C for about 13 minutes. The cookies were cooled. Final weight after baking was about 8.8 g.

A jam topping was prepared by mixing raspberry filling (Sweet Ovations 105182) with sufficient dicalcium phosphate anhydrous (DCPA) (Chemische Fabrik Budenheim) so that the filling was 9.4% DCPA by weight; 5.5 g of this filling was applied to the surface of each cooled cookie. Additional cookies were made separately with the above formula lacking the fiber blend. These cookies were crumbled into coarse crumbs. The crumbs (1.0 g) were applied to the surface of the jam topping of each cookie to prevent sticking. The cookies were packaged in individual foil pouches.

### Example 4

Cookie with Fiber Blend Lacking Pectin

The extruded fiber used in this example was prepared as described in Example 3, except that polydextrose was substituted in the place of inulin. The fiber blend contained low molecular weight alginate (Manugel LBA) and medium molecular weight alginate (Manugel GHB), but lacked pectin. The fiber blend also contained 25% polydextrose (Danisco).

A cookie base was prepared with the following composition:

| | | | % Final | |
|---|---|---|---|---|
| Ingredient | Supplier | % Dough | Formulation | Amount |
| Benefat | Danisco | 18.200 | 10.693 | 72.80 |
| Sugar (Fine | MI Sugar | 18.200 | 10.693 | 72.80 |
| Granulated) | | | | |
| Water | Domestic | 2.900 | 1.704 | 11.60 |
| Molasses, Black | International | 2.400 | 1.410 | 9.60 |
| Strap | Sweeteners | | | |
| Sucralose, 25% | Tate and Lyle | 0.034 | 0.020 | 0.14 |
| Solution | | | | |
| Lecithin Yelkin TM | ADM | 0.750 | 0.441 | 3.00 |
| Vanilla N&A | Firminich | 0.800 | 0.470 | 3.20 |
| 597970 T | | | | |
| Flour, Cookie | Minnel | 13.013 | 7.645 | 52.05 |
| | Milling | | | |
| Baking Soda | Church & | 0.250 | 0.147 | 1.00 |
| | Dwight | | | |
| Quick Rolled Oats | Can-Oat | 18.619 | 10.939 | 74.48 |
| | Milling | | | |
| Fiber Blend | | 21.333 | 12.533 | 85.33 |
| (Manugel LBA | | | | |
| (about 53.7%) and | | | | |
| Manugel GHB | | | | |
| (about 46.3%), | | | | |
| extruded with | | | | |
| polydextrose | | | | |
| (Davisco) to 25% of | | | | |
| final composition | | | | |
| Glycerol, Superol | P&G | 3.500 | 2.056 | 14.00 |
| 99.7% | | | | |
| Total | | 100.00 | 58.75 | 400.00 |

The cookies were prepared and jam topping applied as described in Example 2.

### Example 5

Cookie with Fiber Extruded with Isomalt

The fiber utilized in the example below contained 75% high molecular weight alginate (Manugel DPB) and 25% isomalt (Cargill). The fiber blend was prepared by extrusion as described in Example 3, except that isomalt was substituted in the place of inulin.

A cookie base was prepared with the following composition:

| | | | % Final | |
|---|---|---|---|---|
| Ingredient | Supplier | % Dough | Formulation | Amount |
| Corn Oil | Mazola | 10.500 | 6.17 | 57.75 |
| Vitamin E | Eastman | 0.0036 | 0.002 | 0.020 |
| Sugar (Fine | MI Sugar | 18.200 | 10.693 | 100.10 |
| Granulated) | | | | |
| Water | Domestic | 6.430 | 3.778 | 35.37 |
| Molasses, Black | International | 2.400 | 1.410 | 13.20 |
| Strap | Sweeteners | | | |
| Sucralose, 25% | Tate and Lyle | 0.264 | 0.16 | 1.45 |
| Solution | | | | |
| Lecithin Yelkin TM | ADM | 0.750 | 0.441 | 4.13 |
| Vanilla N&A | Firminich | 0.800 | 0.470 | 4.40 |
| 597970 T | | | | |
| Flour, Cookie | Minnel | 13.800 | 8.108 | 75.90 |
| | Milling | | | |
| Baking Soda | Church & | 0.250 | 0.147 | 1.38 |
| | Dwight | | | |
| Quick Rolled Oats | Can-Oat | 18.786 | 11.037 | 103.32 |
| | Milling | | | |
| Fiber Blend | | 24.316 | 14.286 | 133.73 |
| (Manugel DPB | | | | |
| extruded with | | | | |
| isomalt) | | | | |
| Glycerol, Superol | P&G | 3.500 | 2.056 | 19.25 |
| 99.7% | | | | |
| Total | | 100.00 | 58.75 | 550.00 |

The cookies were prepared and jam topping applied as described in Example 2.

### Example 6

Cookie with Medium Molecular Weight Alginate

The fiber blend of the example below contained medium molecular weight alginate (ISP Manugel GHB) and 25% polydextrose. The blend was prepared by extrusion as described in Example 3, except that polydextrose was substituted in place of inulin.

A cookie base was prepared with the following composition:

| | | % | % Final | |
|---|---|---|---|---|
| Ingredient | Supplier | Dough | Formulation | Amount |
| Soybean Oil | Crisco | 10.500 | 6.170 | 42.00 |
| Vitamin E | Eastman | 0.0036 | 0.002 | 0.0144 |
| Sugar (Fine | MI Sugar | 18.200 | 10.692 | 72.80 |
| Granulated) | | | | |
| Water | Tap | 6.430 | 3.778 | 25.72 |
| Molasses, Black | International | 2.400 | 1.410 | 9.60 |
| Strap | Sweeteners | | | |
| Sucralose, 25% | Tate and | 0.264 | 0.155 | 1.06 |
| Solution | Lyle | | | |
| Lecithin Yelkin TM | ADM | 0.750 | 0.440 | 3.00 |
| Vanilla N&A | Firminich | 0.800 | 0.470 | 3.20 |
| 597970 T | | | | |
| Flour, Cookie | Minnel | 13.800 | 8.110 | 55.20 |
| | Milling | | | |
| Baking Soda | Church & | 0.250 | 0.147 | 1.00 |
| | Dwight | | | |
| Quick Rolled Oats | Can-Oat | 18.786 | 11.034 | 75.14 |
| | Milling | | | |
| Medium Molecular | | 24.316 | 14.286 | 97.26 |
| Weight Alginate | | | | |
| Extruded with | | | | |
| Polydextrose | | | | |
| Glycerol, Superol | P&G | 3.500 | 2.056 | 14.00 |
| 99.7% | | | | |
| Total | | 100.00 | 58.75 | 400.00 |

### Example 7

(a) Determination of Dough Lay Time

When a dough for a cookie or other baked or extruded food product is prepared on a commercial scale, the physical properties of the dough are crucial for acceptable performance in later stages of food processing, particularly depositing through a hopper, during rolling or extrusion of the dough, and cutting or molding of the dough strand or sheet thereby produced into portions for subsequent baking. Dough that is too sticky or viscous will bind to equipment, or rip during processing, preventing the formation of uniform portions for baking. Dough that crumbles readily or lacks sufficient tackiness will resist formation into an acceptable strand or sheet, and portions produced for subsequent baking will form crumbs or not maintain their integrity. The incorporation of substantial amounts of dietary fiber complicates the production of dough with acceptable lay times. In particular, and without being bound by theory, it is believed that fiber tends to hydrate slowly after the dough is formed, and this hydration is accompanied by the decrease in the amount of unbound water in the dough, resulting in a dry dough which crumbles or lacks integrity. Attempts to compensate for this behavior by increasing the initial water content of the dough may result in the dough being initially too sticky, rendering it unacceptable for commercial processes. Dough lay time is a measure, in minutes, of the amount of time after mixing that the dough will maintain acceptable properties for commercial processing. Improvements that improve the dough lay time performance of compositions high in soluble fiber obviously have great commercial value.

The dough lay time test is a subjective cohesiveness rating based on a scale of 1 to 5 (1 is very poor performance; 5 is good performance). Generally, a dough lay score of 4 is needed for acceptable processing on a plant scale, and a score of 4.5 is highly preferred.

The dough lay test is performed by compressing a sample of dough in the hand. The cohesiveness of the dough is evaluated on the above 1-5 scale, with performance of 1 represented by material that is dry, crumbly, and not cohesive. In particular, it does not form a ball when squeezed, or the ball falls apart easily. A performance of 5 is represented by moist, cookie-like dough that compresses when squeezed. A ball formed in the hand remains tightly compressed, and the hand feels moist or sticky thereafter. After a batch of dough is produced, the dough test is performed immediately (0 minute time point), as well as 15, 30, and 45 minutes after production. The amount of time that dough maintains acceptable performance (that is, a score of 4 or greater) is a measure of how much time is available for commercial processing of the dough after it is produced.

7(b) Sensory Evaluation for Slime and Mouth Feel

Incorporation of large amounts of soluble fiber into a product has a detrimental impact on the organoleptic properties. In particular, soluble fiber tends to impart a slimy or viscous property to product during mastication. Without being bound by theory, it is believed that hydrating fiber in the mouth binds to the teeth and the oral soft tissues, resulting in a viscous coating layer that is sensed as a slimy quality. Sliminess of a product is measured by a subjective sensory test. Tooth packing is the adherence of product to the teeth, particularly the crevices in the crowns of the molars, as well as adherence to teeth at the gingival margins (gum packing). Without being bound by theory, it is believed that mastication forces food particles into the crown crevices and gingival margins, and the mass is held in place by the viscous and adhesive properties of the soluble fiber. Creation of products with decreased slime and tooth and gum packing scores is highly preferred, since this will enhance consumer acceptability.

The sensory evaluation is performed by chewing about eight g of the product, and assigning a numeric score to the amount of mouth sliminess or tooth and gum packing sensed after mastication. The product is then expectorated, and the mouth vigorously rinsed with water (about two or three mouthfuls, or about 50 ml). Each mouthful of water is expectorated. The sensory evaluation is then performed again. Tooth and gum packing is similarly evaluated before and after rinsing. A final assessment for slime is performed after approximately three minutes to evaluate a delayed response in the formation of a slime sensation. For samples that produce a large perception of slime, additional evaluations for slime may be performed at five, fifteen, and thirty minutes. A control product is used as standard, and arbitrarily assigned a score of 30 units. If a test product produces a very small difference in slime (either an increase or decrease), it is assigned a score that is 5-6 units different. Therefore, a product that is slightly less slimy than the control may have a score of 25, and a product that is slightly more slimy than the control may have a score of 35. A large difference in slime (either more or less) would be assigned a score 15-20 units different from the control. A huge difference in slime would be reflected in a score that was 30 points different than the control. A similar scale is used for the evaluation of tooth and gum packing. Therefore, an ideal product would have a slime score of about 0 (or a difference of 30 from the control), and a similar score for tooth and gum packing.

7(c) Cookie with Unprocessed High Molecular Weight Alginate

A cookie base was prepared with the following composition:

| | | | % Final | |
|---|---|---|---|---|
| Ingredient | Supplier | % Dough | Formulation | Amount |
| Soybean Oil | Crisco | 10.500 | 6.169 | 42.00 |
| Vitamin E | Eastman | 0.0036 | 0.0021 | 0.0144 |
| Sugar (Fine | MI Sugar | 18.200 | 10.693 | 72.80 |
| Granulated) | | | | |
| Water | Domestic | 8.830 | 5.188 | 35.32 |
| Molasses, Black | International | 2.400 | 1.410 | 9.60 |
| Strap | Sweeteners | | | |
| Sucralose, 25% | Tate and | 0.264 | 0.160 | 1.06 |
| Solution | Lyle | | | |
| Lecithin, Yelkin TM | ADM | 0.750 | 0.441 | 3.00 |
| Vanilla N&A | Firminich | 0.800 | 0.470 | 3.20 |
| 597970 T | | | | |
| Flour, Cookie | Minnel | 17.500 | 10.282 | 70.00 |
| | Milling | | | |
| Baking Soda | Church & | 0.250 | 0.147 | 1.00 |
| | Dwight | | | |
| Quick Rolled Oats | Can-Oat | 18.786 | 11.037 | 75.14 |
| | Milling | | | |
| High Molecular | ISP Manugel | 18.237 | 10.715 | 72.95 |
| Weight Alginate | DPB | | | |
| Glycerol, Superol | P&G | 3.500 | 2.056 | 14.00 |
| 99.7% | | | | |
| Total | | 100.00 | 58.77 | 400.00 |

The cookies were prepared and baked as described in Example 2, but no jam was applied, since the flavor and acidity of the jam tend to interfere with the slime evaluation.

7(d): Cookie with Processed High Molecular Weight Alginate

The fiber used in the following example was extruded as described in Example 3, with polydextrose substituted for inulin.

A cookie base was prepared with the following composition:

| | | | % Final | |
|---|---|---|---|---|
| Ingredient | Supplier | % Dough | Formulation | Amount |
| Soybean Oil | Crisco | 10.500 | 6.17 | 42.00 |
| Vitamin E | Eastman | 0.0036 | 0.002 | 0.0144 |
| Sugar (Fine | MI Sugar | 18.200 | 10.693 | 72.80 |
| Granulated) | | | | |
| Water | Domestic | 6.430 | 3.778 | 25.72 |
| Molasses, Black | International | 2.400 | 1.410 | 9.60 |
| Strap | Sweeteners | | | |
| Sucralose, 25% | Tate and | 0.264 | 0.16 | 1.06 (??) |
| Solution | Lyle | | | |
| Lecithin, Yelkin TM | ADM | 0.750 | 0.441 | 3.00 |
| Vanilla N&A | Firminich | 0.800 | 0.470 | 3.20 |
| 597970 T | | | | |
| Flour, Cookie | Minnel | 13.800 | 8.108 | 55.20 |
| | Milling | | | |
| Baking Soda | Church & | 0.250 | 0.147 | 1.00 |
| | Dwight | | | |
| Quick Rolled Oats | Can-Oat | 18.786 | 11.037 | 75.14 |
| | Milling | | | |
| High Molecular | | 24.316 | 14.286 | 97.26 |
| Weight Alginate | | | | |
| (ISP Manugel | | | | |
| DPB), | | | | |
| extruded with | | | | |
| polydextrose | | | | |
| Glycerol, Superol | P&G | 3.500 | 2.056 | 14.00 |
| 99.7% | | | | |
| Total | | 100.00 | 58.75 | 400.00 |

The cookie formulation was changed slightly from the formulation with unprocessed high molecular weight alginate to order to maintain a consistency suitable for processing and baking. Also, the weight of extruded fiber added was adjusted to compensate for the water content of the extruded fiber. The cookies were prepared and baked as described in Example 2, but no jam was applied.

The two cookie compositions disclosed above were evaluated for dough lay time and slime. Results are presented in the following tables:

Dough Lay Time (Minutes)

| Composition | 0 | 15 | 30 | 45 | Comments |
|---|---|---|---|---|---|
| Cookie with | 7.0 | 6.0 | 6.0 | 5.5 | Cohesive, but |
| Processed High | | | | | sticky soft, |
| Molecular Weight | | | | | slightly oily |
| Alginate | | | | | |
| Cookie with | 7.0 | 6.0 | 4.5 | <4.0 | Dried rapidly |
| Unprocessed High | | | | | |
| Molecular Weight | | | | | |
| Alginate | | | | | |

Slime Evaluation

| | Mouth Coating | | | Tooth Packing | |
|---|---|---|---|---|---|
| | Before | After | Delayed | Before | After |
| Composition | Rinsing | Rinsing | Response | Rinsing | Rinsing |
| Cookie with | 15 | 10 | 20 | 15 | 25 |
| Processed High | | | | | |
| Molecular Weight | | | | | |
| Alginate | | | | | |
| Cookie with | 20 | 25 | 30 | 25-30 | 30 |
| Unprocessed | | | | | |
| High Molecular | | | | | |
| Weight Alginate | | | | | |

The cookie made with the processed fiber composition displayed much improvement in dough lay time, in comparison to the cookie made with the unprocessed control fiber. Importantly, the dough was still processable at 45 minutes, which is important in commercial baking because this permits larger batch sizes, and allows the dough to still be used after brief equipment interruptions.

The cookie made with the processed fiber composition also displayed marked improvements in mouth coating and tooth packing. These improvements are sufficient to make a baked composition acceptable to consumers, whereas a similar composition made with the unprocessed fiber would be viewed as unacceptable.

What is claimed is:

1. An ingestible composition consumed with a meal comprising a combination of effective amounts of at least one soluble anionic fiber and at least one multivalent cation wherein the combination of effective amounts of at least one soluble anionic fiber and at least one multivalent cation reduces the blood glucose response of the meal, in comparison to the blood glucose response to that meal consumed with a composition equicaloric to the ingestible composition but lacking a combination of effective amounts of at least one soluble anionic fiber and at least one multivalent cation.

2. An ingestible composition of claim 1, wherein the composition is produced by a process selected from the group consisting of extrusion, pressing, molding, wire cutting, and mixtures thereof.

3. An ingestible composition of claim 1, wherein the at least one soluble anionic fiber comprises alginate and pectin.

4. An ingestible composition of claim 3, wherein the alginate comprises a medium molecular weight form of alginate and a low molecular weight form of alginate.

5. An ingestible composition of claim 1, wherein the at least one soluble anionic fiber comprises a high molecular weight form of alginate.

6. An ingestible composition of claim 3, wherein a ratio of total alginate to total pectin is from about 8:1 to about 1:8.

7. An ingestible composition of claim 1, wherein the multivalent cation is selected from the group consisting of calcium, magnesium, aluminum, manganese, iron, nickel, copper, zinc, strontium, barium, bismuth, chromium, vanadium, and lanthanum, their salts and mixtures thereof.

8. An ingestible composition of claim 7, wherein the multivalent cation salt is selected from the group of multivalent cation salts consisting of formate, fumarate, acetate, propionate, butyrate, caprylate, valerate, lactate, citrate, malate, gluconate, citrate malate, chloride, phosphate, and mixtures thereof.

9. An ingestible composition of claim 7, wherein the multivalent cation is calcium and wherein the salt is selected from the group consisting of calcium citrate, calcium tartrate, calcium succinate, calcium fumarate, calcium adipate, calcium malate, calcium lactate, calcium gluconate, dicalcium phosphate dihydrate, anhydrous calcium diphosphate, calcium citrate malate, dicalcium phosphate anhydrous, calcium chloride, calcium acetate monohydrate, and mixtures thereof.

10. An ingestible composition of claim 3, wherein the ratio of the at least two soluble anionic fibers to the at least one multivalent cation in the ingestible composition is from about 20:1 to about 7:1.

11. An ingestible composition consumed with a meal comprising a combination of effective amounts of at least one soluble anionic fiber and at least one multivalent cation wherein the combination of effective amounts of at least one soluble anionic fiber and at least one multivalent cation reduces the blood insulin response of the meal, in comparison to the blood insulin response to that meal consumed with a composition equicaloric to the ingestible composition but lacking a combination of effective amounts of at least one soluble anionic fiber and at least one multivalent cation.

12. An ingestible food of claim 11, wherein the formed food product is produced by a process selected from the group consisting of extrusion, pressing, molding, wire cutting, and mixtures thereof.

13. An ingestible composition of claim 11, wherein the at least one soluble anionic fiber comprises alginate and pectin.

14. An ingestible composition of claim 13, wherein the alginate comprises a medium molecular weight form of alginate and a low molecular weight form of alginate.

15. An ingestible composition of claim 11 wherein the at least one soluble anionic fiber comprises a high molecular weight form of alginate.

16. An ingestible composition of claim 13, wherein a ratio of total alginate to total pectin is from about 8:1 to about 1:8.

17. An ingestible composition of claim 11, wherein the multivalent cation is selected from the group consisting of calcium, magnesium, aluminum, manganese, iron, nickel, copper, zinc, strontium, barium, bismuth, chromium, vanadium, and lanthanum, their salts and mixtures thereof.

18. An ingestible composition of claim 17, wherein the multivalent cation salt is selected from the group of multivalent cation salts consisting of formate, fumarate, acetate, propionate, butyrate, caprylate, valerate, lactate, citrate, malate, gluconate, citrate malate, chloride, phosphate, and mixtures thereof.

19. An ingestible composition of claim 17, wherein the multivalent cation is calcium and wherein the salt is selected from the group consisting of calcium citrate, calcium tartrate, calcium succinate, calcium fumarate, calcium adipate, calcium malate, calcium lactate, calcium gluconate, dicalcium phosphate dihydrate, anhydrous calcium diphosphate, calcium citrate malate, dicalcium phosphate anhydrous, calcium chloride, calcium acetate monohydrate, and mixtures thereof.

20. An ingestible composition of claim 11, wherein a ratio of the at least two soluble anionic fibers to the at least one multivalent cation to in the ingestible composition is from about 20:1 to about 7:1.

21. A method of treatment for reducing blood glucose levels after a meal, the method comprising administering an ingestible composition comprising a combination of effective amounts of at least one soluble anionic fiber and at least one multivalent cation wherein the combination of effective amounts of at least one soluble anionic fiber and at least one multivalent cation reduces the blood glucose response of the meal, in comparison to the blood glucose response to that meal consumed with a composition equicaloric to the ingestible composition but lacking a combination of effective amounts of at least one soluble anionic fiber and at least one multivalent cation.

22. The method of claim 21 wherein the ingestible composition is consumed during the consumption of the meal.

23. The method of claim 21 wherein the ingestible composition is consumed prior to consumption of the meal.

24. The method of claim 23 wherein the ingestible composition is consumed from about 90 minutes to about one minute prior to the initiation of consumption of the meal.

25. The method of claim 23 wherein the ingestible composition is consumed from about 30 minutes to about one minute prior to the initiation of the meal.

26. The method of claim 23 wherein the ingestible composition is consumed from about 15 minutes to about one minute prior to initiation of the meal.

27. The method of claim 21 wherein the ingestible composition is consumed after consumption of a meal.

28. The method of claim 27 wherein the ingestible composition is consumed from about one minute to about 90 minutes after completion of consumption of the meal.

29. The method of claim 27 wherein ingestible composition is consumed from about one minute to about 60 minutes after completion of consumption of the meal.

30. The method of claim 27 wherein the ingestible composition is consumed from about one minute to about 30 minutes after completion of consumption of the meal.

31. The method of claim 21, wherein the reduction in blood glucose levels after a meal is from about 5% to about 20%, as measured by the area under the curve over the interval of about 60 minutes after completion of the meal to about 300 minutes after completion of the meal.

32. The method of claim 31, wherein the reduction in blood glucose levels is from about 5% to about 15%.

33. The method of claim 32, wherein the reduction in blood glucose levels is from about 5% to about 10%.

34. A method for reducing postprandial glucose levels in an animal, the method comprising the step of orally administering to the animal a serving of a food comprising a formed solid phase comprising at least one soluble anionic fiber in a total amount of from about 0.5 g to about 10 g per serving and a fluid phase in intimate contact with the formed solid phase, the fluid phase comprising calcium in an amount of from about 50 to about 1000 mg of elemental calcium per serving.

35. A method of treatment for reducing blood insulin levels after a meal, the method comprising administering an ingestible composition comprising a combination of effective amounts of at least one soluble anionic fiber and at least one multivalent cation wherein the combination of effective amounts of at least one soluble anionic fiber and at least one multivalent cation reduces the blood insulin response of the meal, in comparison to the blood insulin response to that meal consumed with a composition equicaloric to the ingestible composition but lacking a combination of effective amounts of at least one soluble anionic fiber and at least one multivalent cation.

36. The method of claim 35 wherein the ingestible composition is consumed during the consumption of the meal.

37. The method of claim 35 wherein the ingestible composition is consumed prior to consumption of the meal.

38. The method of claim 37 wherein the ingestible composition is consumed from about 90 minutes to about one minute prior to the initiation of consumption of the meal.

39. The method of claim 37 wherein the ingestible composition is consumed from about 60 minutes to about one minute prior to the initiation of the meal.

40. The method of claim 37 wherein the ingestible composition is consumed from about 30 minutes to about one minute prior to initiation of the meal.

41. The method of claim 35, wherein the ingestible composition is consumed after the meal.

42. The method of claim 41 wherein the ingestible composition is consumed from about one minute to about 90 minutes after completion of consumption of the meal.

43. The method of claim 41 wherein ingestible composition is consumed from about one minute to about 60 minutes after completion of consumption of the meal.

43. The method of claim 41 wherein the ingestible composition is consumed from about one minute to about 30 minutes after completion of consumption of the meal.

44. The method of claim 35 wherein the reduction in blood insulin levels after a meal is from about 20% to about 40%, as measured by the area under the curve over the interval from about 60 minutes to about 300 minutes after completion of the meal.

45. The method of claim 44 wherein the reduction in blood insulin levels after a meal is from about 23% to about 35%, as measured by the area under the curve over the interval from about 60 minutes to about 300 minutes after completion of the meal.

46. The method of claim 44 wherein the reduction in blood insulin levels after a meal is about 30%, as measured by the area under the curve over interval from about 60 minutes to about 300 minutes after completion of the meal.

47. A method of treating, ameliorating, or slowing the progression of diabetes in an animal with diabetes, the method comprising the step of orally administering to the animal a serving a food comprising a formed solid phase comprising at least one soluble anionic fiber in a total amount of from about 0.5 g to about 10 per serving, and a fluid phase in intimate contact with the formed solid phase, the fluid phase comprising calcium in an amount of from about 50 to about 1000 mg of elemental calcium per serving.

48. A method of decreasing pancreatic insulin demand in an animal with diabetes, the method comprising the step of orally administering to the animal an ingestible composition comprising a formed solid phase comprising at least one soluble anionic fiber in a total amount of from about 0.5 g to about 10 per serving, and a fluid phase in intimate contact with the formed solid phase, the fluid phase comprising calcium in an amount of from about 50 to about 1000 mg of elemental calcium per serving, wherein the ingestible composition is consumed with a meal.

49. A method of treating diabetes, the method comprising identifying an animal in need of improved glucose or insulin response, and administering concurrently with a meal an ingestible composition comprising effective amounts of at least one soluble anionic fiber and at least one multivalent cation wherein the combination of fiber and cation reduces the blood glucose or insulin response to the meal, in comparison to the blood glucose or insulin response to the meal consumed with a composition equicaloric to the ingestible composition but lacking a combination of effective amounts of at least one soluble anionic fiber and at least one multivalent cation.

50. A formed ingestible composition with reduced perceptions of sliminess and toothpacking after mastication, the composition comprising a soluble fiber extruded with a carbohydrate.

51. The ingestible composition of claim 50, wherein the soluble fiber is alginate.

52. The ingestible composition of claim 51, wherein the composition further comprises at least one multivalent cation.

53. The ingestible composition of claim 50, wherein the carbohydrate is selected from the group consisting of polydextrose, inulin, or isomalt.
